(19) United States
(12) Patent Application Publication
(10) Pub. No.: US 2024/0207481 A1
(43) Pub. Date: Jun. 27, 2024

(54) KIT OF GEL-FORMING SOLUTIONS INTENDED FOR PREPARATION OF HYDROGEL BASED ON COVALENTLY CROSSLINKED HYDROXYPHENYL DERIVATIVE OF HYALURONAN FOR PREVENTION OF POSTOPERATIVE COMPLICATIONS RELATED TO FORMATION OF COLORECTAL ANASTOMOSIS, USE OF KIT, METHOD OF PREPARATION OF HYDROGEL AND USE THREREOF

(71) Applicant: CONTIPRO A.S., Dolni Dobrouc (CZ)

(72) Inventors: Jiri PARAL, Vysoka nad Labem (CZ); Martin PRAVDA, Sruby (CZ); Lenka KOVAROVA, Frydek-Mistek, Frydek (CZ); Evgeniy TOROPITSYN, Praha 10, Michle (CZ); Ivana SCIGALKOVA, Olomouc, Neredin (CZ); Vladimir VELEBNY, Zamberk (CZ)

(73) Assignee: CONTIPRO A.S., Dolni Dobrouc (CZ)

(21) Appl. No.: 17/998,473

(22) PCT Filed: May 11, 2021
(86) PCT No.: PCT/CZ2021/050051

§ 371 Date: Nov. 10, 2022

(30) Foreign Application Priority Data
May 12, 2020 (CZ) PV 2020-263

(51) Int. Cl. A61L 26/00; C08L 5/08; C08L 5/16; C12N 9/08

(52) U.S. Cl. CPC A61L 26/008; A61L 26/0052; A61L 26/0066; A61L 26/009; C08L 5/08; C08L 5/16; C12N 9/0065; A61L 2300/406; A61L 2430/34; C12Y 111/01007

(57) ABSTRACT

A kit of gel-forming solutions for a preparation of a biodegradable hydrogel based on a covalently crosslinked hydroxyphenyl derivative of hyaluronan is provided. The kit comprises at least two aqueous solutions, A and B, wherein the solution A comprises horseradish peroxidase and the solution B comprises hydrogen peroxide. At least one of the solution A and/or the solution B comprises a hydroxyphenyl derivative of a hyaluronan, and at least one of the solution A and/or the solution B comprises triclosan and hydroxypropyl-β-cyclodextrin. A method of preparing a hydrogel containing a covalently crosslinked hydroxyphenyl derivative of hyaluronan, from the kit, is also provided. The method comprises preparing separately the at least two solutions A and B, and mixing together the solution A with the solution B to form the hydrogel containing a covalently crosslinked hydroxyphenyl derivative of hyaluronan.

### CROSS-REFERENCE TO RELATED APPLICATIONS

This application is the National Stage of International Application No. PCT/CZ2021/05051, filed on 5 May 2021, which claims priority to and all advantages of CZ Application No. PV2020-263, filed on 12 May 2020, the contents of which are hereby incorporated by reference.

### TECHNICAL FIELD

The present disclosure relates to a kit of gel-forming solutions for a preparation of a hydrogel based on a covalently crosslinked hydroxyphenyl derivative of hyaluronan for the prevention of postoperative complications associated with colorectal anastomosis resulting from anastomic leakage and including, in particular, dehiscence of colorectal anastomosis and development of inflammation. It further relates to an use of the kit, a method of the preparation of the hydrogel and use thereof.

### BACKGROUND

Colorectal carcinoma (CRC) is a disease of affluence. Malignant neoplasm of colorectum is one of the most common oncological diagnoses. The relative five-year survival of patients with colorectal cancer in cases diagnosed in 2001-2005 was about 50% in both sexes (calculated from all reported cases, i.e. treated and for various reasons untreated). In the European Union, the incidence of rectal cancer is from about 15-25 newly diagnosed tumors per population of 100,000 per year. Mortality is reported among from about 4-10 patients per population of 100,000 per year with a slight predominance of the male population.

Procedures used in the surgical treatment of CRC include resection of the affected part of the intestine and subsequent formation of an anastomosis (connection). One of the most serious early complications of this procedure is an anastomic leak, which leads to leakage of the contents of the digestive tract outside the intestinal lumen. The presence of intestinal bacteria in the small pelvic area can cause infection with localized (pelvic abscess) or generalized (peritonitis, sepsis) manifestations. Leakage of the anastomosis can also lead to dehiscence of the anastomosis. It may be a localized problem that does not affect most of the circumference of the anastomosis, but it may also be a complete disintegration of the anastomosis. Such a condition endangers the patient's life, it is necessary to solve it operatively and there is a real risk that the patient will be dependent on an artificial intestinal outlet for the rest of his life.

Among the risk factors for the development of anastomic leakage or dehiscence of the anastomosis includes intestinal ischemia in the suture line, excessive tension in the anastomosis, the presence of local sepsis, etc. Complications arise either due to a poorly technically performed connection (usually within about 48 hours of surgery) or more often from poor healing of the anastomosis. This usually occurs between the about 4^{th} and about 6^{th} postoperative day, with very low rectal resections even later.

There is currently a number of surgical procedures that try to minimize the onset of this complication, yet anastomosis dehiscence occurs in from about 5 to about 20% of operated patients. So-called staplers are used to reconnect the digestive tract, that are designed for fast and even suturing of the tissue. Various types of tissue adhesives are often used as a supplement or replacement for suture material to increase the resistance of gastrointestinal anastomoses. Their task is to strengthen the joint of the digestive tract and their presence is to reduce the leakage of intestinal contents into the peritoneum. Fibrin adhesives, cyanoacrylates, polyethylene glycol-based hydrogels and gelatin-based hydrogels are most commonly used in this indication.

Clinically, fibrin glue is currently the most commonly used to support the healing of anastomosis, the use of which probably has a really positive effect on the healing process of colorectal anastomosis and increase of the resistance of the joint to anastomosis leakage. However, if the intestine is perforated, despite the increased resistance of the anastomosis, e.g. due to its ischemization due to inappropriate surgical techniques, the mere presence of a tissue adhesive does not provide any additional protection against the possible development of infection.

Despite the large number of preclinical studies performed to verify the effectiveness of the use of tissue adhesives in the formation of colorectal anastomosis, their role is not entirely clear. Studies generally agree that the presence of tissue adhesive will reduce unwanted leakage of intestinal contents out of the intestinal lumen and increase the strength of the tissue connection in the short term. However, greater joint rigidity may prevent peristaltic bowel movements, which may increase the risk of bowel obstruction. In addition, the use of cyanoacrylates can negatively affect the healing of the surgical wound. From a longer-term perspective, the use of tissue adhesives, especially cyanoacrylate-based materials, may not be advantageous in a given indication.

Ustek et al. published the results of a preclinical study describing the use of liposomal iodinated povidone (PVP-I) combined with a polyacrylate gel. The publication describes the positive effect of the presence of a hydrogel on the healing of an anastomosis, which is attributed to the combination of wet covering of the inner wound and the broad-spectrum antimicrobial effect of the used PVP-I complex. However, said hydrogel does not provide mechanical support for the anastomosis, nor is mentioned its possible barrier function, preventing the contents of the digestive tract from leaking out of its lumen.

The use of a combination of a hydrogel with fibroblast growth factor or with a platelet-rich plasma (PRP) fraction has also been preclinically tested as a possible way to accelerate the healing of colorectal anastomosis. In both studies, the beneficial effect of increased concentrations of the observed factors on the healing of the anastomosis was described. However, even these hydrogels do not provide protection against the spread of infection in the event of dehiscence of the anastomosis.

A suture material containing the antiseptic triclosan (TCS; e.g. VICRYL® Plus Antibacterial Suture) can currently be used as a means of preventing postoperative infection at the site of the procedure. Indeed, meta-analyzes of the results of clinical studies comparing the incidence of postoperative infections using sutures containing and lacking TCS show a reduction in the likelihood of infection when sutures containing this antimicrobial are used. In the case of colorectal surgery, the included studies described the use of antimicrobial suture material to close the abdominal cavity, resp. suturing of the abdominal fascia.

Triclosan (2,4,4′-trichloro-2′-hydroxydiphenyl ether) is an antimicrobial synthetic substance, poorly soluble in water and well soluble in polar organic solvents (ethanol, chloroform, isopropanol). It is a chemically stable substance that can be stored under normal conditions for many years. Triclosan (trade name Irgasan®, TCS) has been used as an ingredient in a number of cosmetic and pharmaceutical formulations for almost about 50 years. It was originally used as an additive in soaps, shower gels, oral hygiene products, but also as an antiseptic for the production of functional fabrics (surgical gowns) and plastics (kitchen utensils, children's toys, antimicrobial surface treatment of medical devices). Due to its extensive use, TCS has been extensively described in terms of antimicrobial efficacy, acute and chronic toxicity, mutagenicity, reproductive toxicity, and teratogenicity.

Triclosan has a broad spectrum of biocidal activity, which includes Gram positive and Gram negative non-sporulating bacteria, some species of fungi and yeasts. It also has antiviral effects. TCS exhibits both bacteriostatic and bactericidal effects in a concentration-dependent manner. At lower concentrations, the inhibitory effect of TCS on the activity of enoyl-acyl carrier protein (ACP) reductase (FabI), which is a key enzyme for the synthesis of fatty acids in bacteria, is particularly evident. At higher concentrations of TCS, non-specific mechanisms of action of bisphenols, such as damage to membrane integrity, participate on the biocidal effect.

The possibility of incorporating TCS into the hydrogel structure is limited by its low solubility in water. A possible solution is to prepare the inclusion of TCS with cyclodextrins (CD). For example, triclosan containing supramolecular hydrogels based on pluronic acid F-127 and α-cyclodextrin have been prepared by this procedure. Based on structural studies and due to its very favorable pharmacological profile, 2-hydroxypropyl-β-cyclodextrin (HP-β-CD) appears to be the most suitable candidate for the preparation of absorbable hydrogels containing TCS/CD inclusion. US20170281781A1 describes that cyclodextrins are able to interact with mucosal surface proteins and the presence of water-soluble cyclodextrin derivatives increases the mucoadhesive properties of hydrogels.

Hyaluronan is a polysaccharide that includes disaccharide units composed of D-glucuronic acid and D-N-acetylglucosamine linked by alternating β-1,4 and β-1,3 glycosidic bonds. The weight average molecular weight (if molecular weight is mentioned below, it will always be the weight average molecular weight) in vivo is in the range of from about 3,000 g/mol to about 20,000,000 g/mol. It is a polysaccharide that is easily soluble in aqueous media, where, depending on molecular weight and concentration, it forms very viscous solutions. Hyaluronan is a component of almost all tissues and body fluids of vertebrates, and is abundant, especially in connective tissues. It is a highly hygroscopic molecule, hyaluronan solutions are strongly osmotically active and the presence of hyaluronan is, among other things, important for tissue hydration.

In addition, hyaluronan is able to modulate inflammatory responses of tissues, both by influencing the production of cytokines and by its effect on the adhesion of cytokine-activated lymphocytes. Its antioxidant properties and ability to scavenge free radicals reduce the activity of proteinases acting during inflammation, whereby hyaluronan contributes to the stabilization of the affected tissue and promotes its granulation.

In addition to their use in the treatment of chronic wounds, hyaluronan-based products are widely used in the prevention of postoperative adhesions. Hyaluronan solutions are used to fill the abdominal cavity after surgery. The presence of the hyaluronan solution is intended to mechanically separate the traumatized surfaces of the internal organs and thus prevent their adhesions. The disadvantage of using these solutions is the short biological half-life of unmodified hyaluronan, which does not provide long-lasting protection against adhesions. In order to solve this problem, hyaluronan containing gels and foils have been proposed in the past, which at the site of action act as a barrier against the formation of adhesions for a longer period of time.

Various types of hyaluronan derivatives have also been developed in the past that are able to undergo a sol-gel transition under physiological conditions in situ. Phenolic hyaluronan derivatives, for example, can be used for this purpose. Calabro et al. describe in EP1587945B1 and EP1773943B1 a method for the preparation of hydroxyphenyl derivatives of hyaluronan by the reaction of carboxyls present in the structure of D-glucuronic acid of hyaluronan, with aminoalkyl derivatives of phenol, e.g. tyramine. Products of this reaction are hyaluronan amides. The same document also discloses that crosslinking of hydroxyphenyl derivatives of hyaluronan can be initiated by the addition of peroxidase (e.g. horseradish peroxidase) and diluted hydrogen peroxide solution. Horseradish peroxidase (HRP, E.C.1.11.1.7) is currently widely used as a catalyst for organic and biotransformation reactions. Hydrogels based on hydroxyphenyl derivatives of hyaluronan can be used as injectable matrices for controlled release of substances or as materials suitable for culturing and implanting cells. WO/2017/197262 describes the use of a tyraminated hyaluronan derivative for the preparation of a hydrogel matrix containing several types of reservoirs of biologically active substances. It also uses a horseradish peroxidase-mediated reaction to crosslink the hydrogel. Wolfova et al. disclose in CZ303879 a conjugate of hyaluronan and tyramine comprising an aliphatic linker inserted between a polymer chain and tyramine. The presence of an aliphatic linker allows a higher efficiency of the crosslinking reaction and gives the network higher elasticity. This document also describes the possible use of hydrogels based on the respective derivative as a biodegradable barrier preventing the formation of postoperative adhesions.

Although hyaluronan is widely used as a material for the prevention of postoperative adhesions, in the case of prevention of complications associated with colorectal anastomosis, where there is an increased risk of infection and development of peritonitis, the use of materials based on it is not always advantageous. Hyaluronan hydrogel crosslinked with polyvalent iron ions (Intergel) has been evaluated in the past as a material that increases the virulence of some bacterial strains and increases the risk of postoperative complications. Antiadhesive membranes based on a mixture of hyaluronan and carboxymethylcellulose are contraindicated when used in direct contact with intestinal anastomoses, for direct contact with the suture line of the anastomosis and in the case of clinically manifested infection.

### SUMMARY

A kit of gel-forming solutions for a preparation of a biodegradable hydrogel based on a covalently crosslinked hydroxyphenyl derivative of hyaluronan is provided. The kit comprises at least two aqueous solutions, A and B, wherein the solution A comprises horseradish peroxidase and the solution B comprises hydrogen peroxide. At least one of the solution A and/or the solution B comprises a hydroxyphenyl derivative of a hyaluronan having the general formula I:

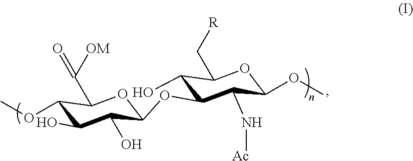

wherein n is from 2 to 5000, each M is H+ or a cation of a pharmaceutically acceptable salt selected from the group of alkali metal cations and alkaline earth metal cations, and each R is independently —OH or a substituent of having the general formula II:

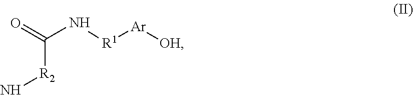

where Ar is phenylene and R1 is ethylene, or Ar is indolydene and R1 is ethylene, or Ar is hydroxyphenylene and R1 is carboxyethylene, and where R2 is an alkylene group having from 3 to 7 carbon atoms; and wherein the solution A and/or the solution B comprises triclosan and hydroxypropyl-β-cyclodextrin.

A method of preparing a hydrogel containing a covalently crosslinked hydroxyphenyl derivative of hyaluronan is also provided. The hydrogel is intended for prevention of postoperative complications associated with colorectal anastomosis, and utilizes the kit as set forth above. Specifically, the method comprises preparing separately the at least two solutions A and B; and mixing together the solution A with the solution B to form the hydrogel containing a covalently crosslinked hydroxyphenyl derivative of hyaluronan.

### BRIEF DESCRIPTION OF THE DRAWINGS

FIG. 1: The graph shows the differences in the rate of weight loss of hydrogels upon degradation by hyaluronidase depending on the efficiency of their crosslinking.

FIG. 2: Antimicrobial effect of test samples on the bacterial strain Staphylococcus aureus.

FIG. 3: Antimicrobial effect of test samples on the bacterial strain Escherichia coli.

FIG. 4: Antimicrobial effect of test samples on the yeast Candida albicans.

FIG. 5: Antimicrobial effect of test samples on the yeast Clostridium sporogenes.

FIG. 6:6A—a prototype of a device including the solution A and the solution B; 6B—colorectal anastomosis in a porcine model; 6C—porcine small pelvis filled with hydrogel

FIG. 7: Application of hydrogel into a porcine small pelvis

FIG. 8: Influence of HP-β-CD on the preparation of gels based on HA-TA

FIG. 9: Change in elastic and viscous module of the gel during gelation.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The object of the present disclosure is to overcome the shortcomings of the prior art and to develop a feature for the preparation of a biodegradable hydrogel based on hyaluronan containing an antiseptic agent in order to prevent postoperative complications associated with the formation of colorectal anastomosis. Such feature is a kit of gel-forming solutions intended for the preparation of a hydrogel based on a covalently crosslinked hydroxyphenyl derivative of hyaluronan, which essentially comprises at least two aqueous solutions A and B, of which the solution A contains horseradish peroxidase and the solution B contains hydrogen peroxide, the solution A and/or the solution B containing a hydroxyphenyl derivative of a hyaluronan of a general formula I

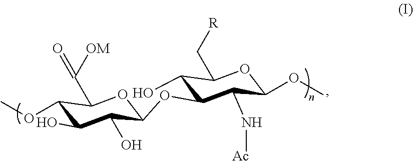

wherein n is in the range of from about 2 to about 5000, M is H⁺ or a cation of a pharmaceutically acceptable salt selected from a group containing alkali metal cation, alkaline earth metal cation, and wherein R is OH or substituent NHR₂CONHR₁ArOH of a general formula II,

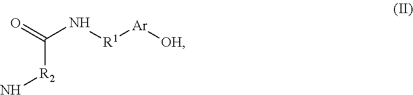

wherein Ar is phenylene and R₁ is ethylene, or Ar is indolydene and R₁ is ethylene, or Ar is hydroxyphenylene and R₁ is carboxyethylene, and R₂ is alkylene of 3 to 7 carbons, and at the same time the solution A and/or the solution B contains triclosan and hydroxypropyl-β-cyclodextrin. The cation of the pharmaceutically acceptable salt is preferably selected from the group containing of Na⁺, K⁺, Mg²⁺ or Li⁺.

Preferably, in the kit of the disclosure, the horseradish peroxidase activity is in the range of from about 0.5 to about 1.5 U/mL, preferably from about 0.9 to about 1.35 U/mL, more preferably from about 0.8 to about 1.2 U/mL, the concentration of hydrogen peroxide is in the range of from about 1 to about 6 mmol/L, preferably from about 3 to about 5 mmol/L, the hydroxyphenyl derivative of hyaluronan according to the general formula I has a weight average molecular weight in the range of from about 60,000 g/mol to about 2,000,000 g/mol, preferably from about 100,000 g/mol to about 1,000,000 g/mol, more preferably from about 200,000 g/mol to about 400,000 g/mol; a degree of substitution in the range of from about 1% to about 10%, preferably from about 1% to about 5%, more preferably from about 2% to about 4% and a concentration of from about 10 to about 50 mg/mL, preferably from about 15 to about 25 mg/mL, more preferably about 20 mg/mL; and the concentration of triclosan is in the range of from about 0.1 to about 2.2 mg/mL, preferably from about 1 to about 2.2 mg/mL, more preferably about 2 mg/mL and the concentration of hydroxypropyl-β-cyclodextrin is in the range of from about 4 to about 100 mg/mL, preferably from about 25 to about 80 mg/mL, more preferably about 60 mg/mL, wherein the molar ratio of triclosan to hydroxypropyl-β-cyclodextrin is in the range of from about 1:4 to about 1:10, preferably in the range of from about 1:5 to about 1:8, more preferably about 1:6.

Polysaccharides, including hyaluronan and derivatives prepared thereof, belong to polymers formed by a mixture of macromolecules of different lengths and thus form non-uniform (polydisperse) systems. The molar mass of such polymers can be expressed as numerical average molar mass (Mn) or weight average molar mass (Mw). The ratio of these two types of average molecular weights of the polymer chains (Mw/Mn) expresses the degree of non-amorphous (polydispersity) of the polymer sample and is referred to as the polydispersity index (PI). The PI of hydroxyphenyl derivative of hyaluronan of the general formula (I) as mentioned above is in the range of from about 1 to about 3.

More preferably, the kit of the disclosure comprises the solution A, which contains:
A hydroxyphenyl derivative of hyaluronan according to the general formula I with a weight-average molar mass in the range of from about 60,000 g/mol to about 2,000,000 g/mol and a polydispersity index of from about 1 to about 3, with a degree of substitution in the range of from about 1% to about 10%, at a concentration from about 10 to about 50 mg/mL,
Horseradish peroxidase with an activity of from about 0.5 to about 1.5 U/mL,
Triclosan, which is at a concentration of from about 0.2 to about 2.2 mg/mL, and
Hydroxypropyl-β-cyclodextrin, which is in a concentration of from about 4 to about 100 mg/mL, wherein the molar ratio of triclosan to hydroxypropyl-β-cyclodextrin is in the range of from about 1:4 to about 1:10;
   and the solution B, which contains:
Hydroxyphenyl derivative of hyaluronan according to the general formula I with a weight average molar weight in the range of from about 60,000 g/mol to about 2,000,000 g/mol and with a polydispersity index of from about 1 to about 3, with a degree of substitution in the range of from about 1% to about 10%, in a concentration of from about 10 to about 50 mg/mL, and
Hydrogen peroxide, in the concentration range of from about 1 to about 6 mmol/L.

Further, more preferably, the kit of the disclosure comprises the solution A, which comprises:
Hydroxyphenyl derivative of hyaluronan according to the general formula I with a weight average molar weight in the range of from about 100,000 g/mol to about 1,000,000 g/mol and with a polydispersity index of from about 1 to about 3, with a degree of substitution in the range of from about 1% to about 5%, whereas it is at concentration of from about 15 to about 25 mg/mL,
Horseradish peroxidase with an activity of from about 0.9 to about 1.35 U/mL,
Triclosan at a concentration of from about 1 to about 2.2 mg/mL,
Hydroxypropyl-β-cyclodextrin at a concentration of from about 25 to about 80 mg/mL, and wherein the molar ratio of triclosan to hydroxypropyl-β-cyclodextrin is in the range of from about 1:5 to about 1:8;
   and the solution B, which contains:
Hydroxyphenyl derivative of hyaluronan according to the general formula I with a weight average molar mass in the range of from about 100,000 g/mol to about 1,000,000 g/mol and with a polydispersity index of from about 1 to about 3, with a degree of substitution in the range of from about 1% to about 5%, whereas it is at concentration from about 15 to about 25 mg/mL, and
Hydrogen peroxide, which is in the concentration range of from about 3 to about 5 mmol/L.

In some embodiments, the kit of the disclosure comprises the solution A, which comprises:
Hydroxyphenyl derivative of hyaluronan according to the general formula I with weight average molar mass in the range of from about 200,000 g/mol to about 400,000 g/mol and with a polydispersity index of from about 1 to about 3, with a degree of substitution in the range of from about 2% to about 4%, at a concentration of about 20 mg/mL,
Horseradish peroxidase with an activity of from about 0.8 to about 1.2 U/mL,
Triclosan at a concentration of about 2 mg/mL, and
Hydroxypropyl-β-cyclodextrin, which is at a concentration of about 60 mg/mL; and the solution B, which contains:
Hydroxyphenyl derivative of hyaluronan according to the general formula I with a weight average molar mass in the range of from about 200,000 g/mol to about 400,000 g/mol and with a polydispersity index of from about 1 to about 3, with a degree of substitution in the range of from about 2% to about 4%, at a concentration of about 20 mg/mL, and
Hydrogen peroxide in the concentration range of from about 4 to about 5 mmol/L.

According to yet another preferred embodiment of the disclosure, the kit comprises aqueous solutions A, B and at least one solution C, wherein the solution A comprises horseradish peroxidase, a hydroxyphenyl derivative of hyaluronan of the general formula I as defined above, triclosan and hydroxypropyl-β-cyclodextrin, the solution B comprises hydrogen peroxide and the solution C comprises a hydroxyphenyl derivative of a hyaluronan of the general formula I as defined above.

Further preferably, the horseradish peroxidase activity in the solution A is in the range of from about 1 to about 3 U/mL, preferably from about 1.6 to about 2.7 U/mL, more preferably from about 1.8 to about 2.4 U/mL, the concentration of hydrogen peroxide in the solution B is in the range of from about 2 to about 12 mmol/L, preferably from about 6 to about 10 mmol/L, a hydroxyphenyl derivative of hyaluronan according to the general formula I that has a weight average molar weight in the range of from about 60,000 g/mol to about 2,000,000 g/mol, preferably from about 100,000 g/mol to about 1,000,000 g/mol, more preferably from about 200,000 g/mol to about 400,000 g/mol; the degree of substitution in the range of from about 1% to about 10%, preferably from about 1% to about 5%, more preferably from about 2% to about 4%, is present in the solution A in a concentration of from about 1 to about 20 mg/mL, preferably from about 5 to about 15 mg/mL, more preferably about 10 mg/mL, and in the solution C at a concentration of from about 10 to about 50 mg/mL, preferably from about 30 to about 40 mg/mL, more preferably about 35 mg/mL, the concentration of triclosan in the solution A is in the range of from about 0.2 to about 4.4 mg/mL, preferably from about 2 to about 4.4 mg/mL, more preferably about 4 mg/mL, the concentration of hydroxypropyl-β-cyclodextrin in the solution A is in the range of from about 8 to about 200 mg/mL, preferably from about 100 to about 160 mg/mL, more preferably about 120 mg/mL, wherein the molar ratio of triclosan to hydroxypropyl-β-cyclodextrin is in the range of from about 1:4 to about 1:10, preferably in the range of from about 1:5 to about 1:8, more preferably about 1:6.

Further, more preferably, the kit of the disclosure comprises the solution A, which comprises:
Hydroxyphenyl derivative of hyaluronan according to the general formula I with a weight-average molar mass in the range of from about 60,000 g/mol to about 2,000,000 g/mol and a polydispersity index of from about 1 to about 3, with a degree of substitution in the range of from about 1% to about 10%, at a concentration from about 1 to about 20 mg/mL,
Horseradish peroxidase with an activity of from about 1 to about 3 U/mL,
Triclosan at a concentration of from about 0.4 to about 4.4 mg/mL, and
Hydroxypropyl-β-cyclodextrin, which is in a concentration of from about 8 to about 200 mg/mL, wherein the molar ratio of triclosan to hydroxypropyl-β-cyclodextrin is in the range of from about 1:4 to about 1:10;
   the solution B, which contains:
Hydrogen peroxide, whereas the concentration is in the range from about 2 to about 12 mmol/L; and
   the solution C, which contains:
Hydroxyphenyl derivative of hyaluronan according to the general formula I with a weight average molar weight in the range of from about 60,000 g/mol to about 2,000,000 g/mol and with a polydispersity index of from about 1 to about 3, with a degree of substitution in the range of from about 1% to about 10%, in a concentration of from about 10 to about 50 mg/mL,

Further, more preferably, the kit of the disclosure comprises the solution A, which comprises:
Hydroxyphenyl derivative of hyaluronan according to the general formula I with a weight average molar mass in the range of from about 200,000 g/mol to about 400,000 g/mol and with a polydispersity index of from about 1 to about 3, with a degree of substitution in the range of from about 2% to about 4%, in a concentration of from about 5 to about 15 mg/mL,
Horseradish peroxidase with an activity of from about 1.6 to about 2.7 U/mL,
Triclosan, which is at a concentration of from about 3.6 to about 4.4 mg/mL,
Hydroxypropyl-β-cyclodextrin, which is in a concentration of from about 108 to about 132 mg/mL, wherein the molar ratio of triclosan to hydroxypropyl-β-cyclodextrin is in the range of from about 1:4 to about 1:10;
   the solution B, which contains:
Hydrogen peroxide in the concentration range of from about 7 to about 12 mmol/L; and the solution C, which contains:
Hydroxyphenyl derivative of hyaluronan according to the general formula I with a weight average molar mass in the range of from about 60,000 g/mol to about 2,000,000 g/mol and with a polydispersity index of from about 1 to about 3, with a degree of substitution in the range of from about 1% to about 10%, in a concentration of from about 30 to about 40 mg/mL.
   And even more preferably, the kit of the disclosure comprises the solution A, which comprises:
Hydroxyphenyl derivative of hyaluronan according to the general formula I with a weight average molar mass in the range of from about 200,000 g/mol to about 400,000 g/mol and with a polydispersity index of from about 1 to about 3, with a degree of substitution in the range of from about 2% to about 4%, at a concentration of about 10 mg/mL,
Horseradish peroxidase with an activity of from about 1.8 to about 2.4 U/mL,
Triclosan, which is at a concentration of from about 3.6 to about 4.4 mg/mL,
Hydroxypropyl-β-cyclodextrin, which is in a concentration of from about 108 to about 132 mg/mL, wherein the molar ratio of triclosan to hydroxypropyl-β-cyclodextrin is in the range of from about 1:4 to about 1:10;
   the solution B, which contains:
Hydrogen peroxide in the concentration range of from about 7 to about 12 mmol/L; and the solution C, which contains:
Hydroxyphenyl derivative of hyaluronan according to the general formula I with a weight average molecular weight in the range of from about 60,000 g/mol to about 2,000,000 g/mol and with a polydispersity index of from about 1 to about 3, with a degree of substitution in the range of from about 1% to about 10%, in a concentration of about 35 mg/mL.

Furthermore, the disclosure also relates to the use of a kit as contemplated herein for the preparation of a biodegradable hydrogel based on a covalently crosslinked hydroxyphenyl derivative of hyaluronan at its site of action in the small pelvic region, where the formed hydrogel serves as prevention of postoperative complications related to formation of colorectal anastomosis. Postoperative complications associated with the formation of a colorectal anastomosis are selected from the group containing of anastomic leakage, opening (dehiscence) of the colorectal anastomosis, development of infection in the small pelvis and peritoneum.

A method of preparation of a hydrogel comprising a covalently crosslinked hydroxyphenyl hyaluronan derivative, wherein at least two solutions A and B as defined in claim 1 are prepared separately, wherein the solution A and/or the solution B comprises a hydroxyphenyl hyaluronan derivative of the general formula I as defined above and, at the same time, the solution A and/or the solution B contains triclosan and hydroxypropyl-β-cyclodextrin, after which the solution A is mixed with the solution B to form a hydrogel containing a covalently crosslinked hydroxyphenyl derivative of hyaluronan, which is intended to prevent postoperative complications associated with colorectal anastomosis.

Another embodiment of the disclosure is a method of preparation of a hydrogel comprising a covalently crosslinked hydroxyphenyl derivative of hyaluronan, whose essence lies in that at least two solutions A and B are prepared separately as described above, wherein the solution A and/or the solution B comprises a hydroxyphenyl derivative of hyaluronan of the general formula I as defined above, and at the same time the solution A and/or the solution B contains triclosan and hydroxypropyl-β-cyclodextrin, after which the solution A is mixed with the solution B to form a hydrogel containing a covalently crosslinked hydroxyphenyl derivative of hyaluronan, which is intended to prevent postoperative complications associated with colorectal anastomosis.

Preferably, the solution A contains horseradish peroxidase with an activity in the range of from about 0.5 to about 1.5 U/mL and the solution B contains hydrogen peroxide in a concentration in the range of from about 1 to about 6 mmol/L, wherein the solution A and/or the solution B contains a hydroxyphenyl derivative of hyaluronan of the general formula I as described above, wherein its weight average molar weight is in the range of from about 60,000 g/mol to about 2,000,000 g/mol, the degree of substitution is in the range of from about 1% to about 10%, and its concentration is in the range of from about 10 to about 50 mg/mL, while the solution A and/or the solution B contains triclosan and hydroxypropyl-β-cyclodextrin, after which the solution A is mixed with the solution B to form a hydrogel containing a covalently cross-linked hydroxyphenyl derivative of hyaluronan, which is intended to prevent postoperative complications associated with the formation of colorectal anastomosis.

Postoperative complications associated with the formation of colorectal anastomosis are selected from the group comprising of colorectal anastomosis disintegration, anastomic leakage, spread of infection.

In a preferred embodiment of the method as contemplated herein, the horseradish peroxidase activity is in the range of from about 0.5 to about 1.5 U/mL, preferably from about 0.9 to about 1.35 U/mL, more preferably from about 0.8 to about 1.2 U/mL, the hydrogen peroxide concentration is in the range of from about 1 to about 6 mmol/L, preferably from about 3 to about 5 mmol/L, the hydroxyphenyl derivative of hyaluronan according to the general formula I has a weight average molar weight in the range of from about 60,000 g/mol to about 2,000,000 g/mol, preferably from about 100 000 g/mol to about 1,000,000 g/mol, more preferably from about 200,000 g/mol to about 400,000 g/mol; a degree of substitution in the range of from about 1% to about 10%, preferably from about 1% to about 5%, more preferably from about 2% to about 4% and a concentration of from about 10 to about 50 mg/mL, preferably from about 15 to about 25 mg/mL, more preferably about 20 mg/mL; and the concentration of triclosan is in the range of from about 0.2 to about 2.2 mg/mL, preferably from about 1 to about 2.2 mg/mL, more preferably about 2 mg/mL and the concentration of hydroxypropyl-β-cyclodextrin is in the range of from about 4 to about 100 mg/mL, preferably from about 25 to about 80 mg/mL, more preferably about 60 mg/mL, wherein the molar ratio of triclosan to hydroxypropyl-β-cyclodextrin is in the range of from about 1:4 to about 1:10, preferably in the range of from about 1:5 to about 1:8, more preferably about 1:6.

According to yet another preferred embodiment of the method as contemplated herein, the solution A comprises:
Hydroxyphenyl derivative of hyaluronan according to the general formula I with a weight average molar weight in the range of from about 60,000 g/mol to about 2,000,000 g/mol and a polydispersity index of from about 1 to about 3, with a degree of substitution in the range of from about 1% to about 10%, at a concentration of from about 10 to about 50 mg/mL,
Horseradish peroxidase with an activity of from about 0.5 to about 1.5 U/mL,
Triclosan, that is at a concentration of from about 0.2 to about 2.2 mg/mL, and
Hydroxypropyl-β-cyclodextrin at a concentration of from about 4 to about 100 mg/mL, wherein the molar ratio of triclosan to hydroxypropyl-β-cyclodextrin is in the range of from about 1:4 to about 1:10;
   and the solution B contains:
Hydroxyphenyl derivative of hyaluronan according to the general formula I with weight average molar mass in the range of from about 60,000 g/mol to about 2,000,000 g/mol and with a polydispersity index of from about 1 to about 3, with a degree of substitution in the range of from about 1% to about 10%, in a concentration of from about 10 to about 50 mg/mL, and
Hydrogen peroxide, in the concentration range of from about 1 to about 6 mmol/L.

According to yet another preferred embodiment of the method as contemplated herein, the solution A comprises:
Hydroxyphenyl derivative of hyaluronan according to the general formula I with a weight average molar weight in the range of from about 100,000 g/mol to about 1,000,000 g/mol and with a polydispersity index of from about 1 to about 3, with a degree of substitution in the range of from about 1% to about 5%, at a concentration of from about 15 to about 25 mg/mL,
Horseradish peroxidase with an activity of from about 0.9 to about 1.35 U/mL,
Triclosan at a concentration of from about 1 to about 2.2 mg/mL, and
Hydroxypropyl-β-cyclodextrin at a concentration of from about 25 to about 80 mg/mL, wherein the molar ratio of triclosan to hydroxypropyl-β-cyclodextrin is in the range of from about 1:5 to about 1:8;
   and the solution B contains:
Hydroxyphenyl derivative of hyaluronan according to the general formula I with a weight average molar mass in the range of from about 100,000 g/mol to about 1,000,000 g/mol and with a polydispersity index of from about 1 to about 3, with a degree of substitution in the range of from about 1% to about 5%, at a concentration of from about 15 to about 25 mg/mL,
Hydrogen peroxide, which is in the concentration range of from about 3 to about 5 mmol/L.
   According to yet another preferred embodiment of the method as contemplated herein, the solution A comprises:
Hydroxyphenyl derivative of hyaluronan according to the general formula I with weight average molar mass in the range of from about 200,000 g/mol to about 400,000 g/mol and with a polydispersity index of from about 1 to about 3, with a degree of substitution in the range of from about 2% to about 4%, at a concentration of about 20 mg/mL,
Horseradish peroxidase with an activity of from about 0.8 to about 1.2 U/mL,
Triclosan at a concentration of about 2 mg/mL,
Hydroxypropyl-β-cyclodextrin, which is at a concentration of about 60 mg/mL; and the solution B contains:
Hydroxyphenyl derivative of hyaluronan according to the general formula I with a weight average molar mass in the range of from about 200,000 g/mol to about 400,000 g/mol and with a polydispersity index of from about 1 to about 3, with a degree of substitution in the range of from about 2% to about 4%, at a concentration of about 20 mg/mL and
Hydrogen peroxide in the concentration range of from about 4 to about 5 mmol/L.

According to yet another preferred embodiment of the method as contemplated herein, the solution A is mixed with the solution B in a volume ratio of about 1:1.

Yet another preferred embodiment of the method as contemplated herein is to prepare solutions A, B and at least one solution C, defined above for a kit comprising solutions A, B and at least one solution C, wherein the solution A comprises horseradish peroxidase, a hydroxyphenyl derivative of hyaluronan of the general formula I, as defined above, triclosan and hydroxypropyl-β-cyclodextrin, the solution B contains hydrogen peroxide and the solution C contains a hydroxyphenyl derivative of hyaluronan of the general formula I as defined above, whereupon solutions A, B and C are mixed to form a hydrogel containing covalently crosslinked hydroxyphenyl derivative of hyaluronan, which is intended to prevent postoperative complications associated with the formation of colorectal anastomoses.

Further preferably, the horseradish peroxidase activity in the solution A is in the range of from about 1 to about 3 U/mL, preferably from about 1.6 to about 2.7 U/mL, more preferably from about 1.8 to about 2.4 U/mL, the concentration of hydrogen peroxide in the solution B is in the range of from about 2 to about 12 mmol/L, preferably from about 6 to about 10 mmol/L, a hydroxyphenyl derivative of hyaluronan according to the general formula I having a weight average molar weight in the range of from about 60,000 g/mol to about 2,000,000 g/mol, preferably from about 100,000 g/mol to about 1,000,000 g/mol, more preferably from about 200,000 g/mol to about 400,000 g/mol; the degree of substitution in the range of from about 1% to about 10%, preferably from about 1% to about 5%, more preferably from about 2% to about 4%, is present in the solution A in a concentration of from about 1 to about 20 mg/mL, preferably from about 5 to about 15 mg/mL, more preferably about 10 mg/mL, and in the solution C at a concentration of from about 10 to about 50 mg/mL, preferably from about 30 to about 40 mg/mL, more preferably about 35 mg/mL, the concentration of triclosan in the solution A is in the range of from about 0.2 to about 4.4 mg/mL, preferably from about 2 to about 4.4 mg/mL, more preferably about 4 mg/mL, the concentration of hydroxypropyl-β-cyclodextrin in the solution A is in the range of from about 8 to about 200 mg/mL, preferably from about 100 to about 160 mg/mL, more preferably about 120 mg/mL, wherein the molar ratio of triclosan to hydroxypropyl-β-cyclodextrin is in the range of from about 1:4 to about 1:10, preferably in the range of from about 1:5 to about 1:8, more preferably about 1:6.

Preferably, solutions A, B, C are mixed in a volume ratio of about 1:1:2.

Yet another embodiment of the disclosure is a hydrogel preparable by the method of the disclosure as set forth above, comprising covalently crosslinked hydroxyphenyl derivative in a concentration of from about 10 to about 50 mg/mL, which is formed by crosslinking a hydroxyphenyl derivative of hyaluronan of the general formula I

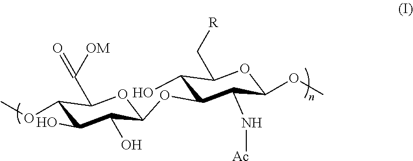

wherein n is in the range of from about 2 to about 5000, M is H⁺ or a cation of a pharmaceutically acceptable salt selected from the group containing of an alkali metal cation, alkaline earth metal cation, and wherein R is OH or substituent NHR₂CONHR₁ArOH of the general formula II,

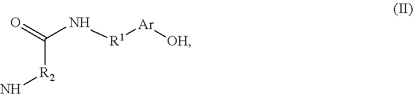

wherein Ar is phenylene and R₁ is ethylene, or Ar is indolydene and R₁ is ethylene, or Ar is hydroxyphenylene and R₁ is carboxyethylene, and R₂ is alkylene of 3 to 7 carbons. In some such embodiments, after mixing the at least two solutions A and B it reaches a gelation point within from about 5 to about 70 s, preferably from about 15 to about 60 s, more preferably from about 25 to about 50 s, while the value of its elastic module reaches from about 100 to about 1000 Pa, no later than about 3 min after mixing the solutions preferably from about 100 to about 600 Pa, more preferably from about 100 to about 500 Pa, and upon completion of the solidification process, its elastic module is in the range of from about 500 to about 2000 Pa, preferably from about 600 to about 1300 Pa, more preferably from about 700 to about 1200 Pa.

The cation of the pharmaceutically acceptable salt is preferably selected from the group containing of Na⁺, K⁺, Mg²⁺ or Li⁺.

Preferably, the hydrogel of the disclosure comprises:
Covalently crosslinked hydroxyphenyl derivative from about 10 to about 50 mg/mL
Horseradish peroxidase from about 0.25 to about 0.75 U/mL
Triclosan from about 0.1 to about 1.1 mg/mL
Hydroxypropyl-β-cyclodextrin from about 2 to about 50 mg/mL.

More preferably, the molar ratio of triclosan to hydroxypropyl-β-cyclodextrin (TCS HP-β-CD ratio) is in the range of from about 1:4 to about 1:10, whereas it reaches the gelation point within from about s to about 70 s after mixing solutions A and B, furthermore, no later than about 3 minutes after mixing the solutions, the value of its elastic module reaches from about 100 to about 1000 Pa, and after the completion of the solidification process, its elastic module is in the range of from about 500 to about 2000 Pa.

More preferably, the hydrogel of the disclosure comprises:
Covalently crosslinked hydroxyphenyl derivative at a concentration of from about 15 to about 25 mg/mL,
Horseradish peroxidase with an activity of from about 0.45 to about 0.675 U/mL,
Triclosan at a concentration of from about 0.5 to about 1.1 mg/mL,
Hydroxypropyl-β-cyclodextrin at a concentration of from about 12 to about 38 mg/mL.

Even more preferably, the molar ratio of triclosan to hydroxypropyl-β-cyclodextrin (TCS: HP-β-CD ratio) is in the range of from about 1:5 to about 1:8, whereas it reaches the gelation point within from about 15 s to about 60 s after mixing solutions A and B, furthermore, no later than about 3 minutes after mixing the solutions the value of its elastic module reaches from about 100 to about 600 Pa and after the completion of the solidification process its elastic module is in the range of from about 600 to about 1300 Pa

Even more preferably, the hydrogel of the disclosure comprises:
Covalently cross-linked hydroxyphenyl derivative at a concentration of about 20 mg/mL,
Horseradish peroxidase with an activity of from about 0.4 to about 0.6 U/mL,
Triclosan at a concentration of about 1 mg/mL,
Hydroxypropyl-β-cyclodextrin at a concentration of about 30 mg/mL.

Even more preferably, after mixing solutions A and B it reaches the gelation point within from about 25 s to about 50 s, furthermore, within about 3 min after mixing the solutions the value of its elastic module reaches from about 100 to about 500 Pa and after completion of the solidification process, its elastic module is in the range of from about 600 to about 1200 Pa.

According to yet another preferred embodiment of the disclosure, the hydrogel as contemplated herein is used as a filling material around the colorectal anastomosis to prevent colorectal anastomosis from opening, anastomic leakage and the spread of infection due to anastomic leakage.

The hydrogel formed using the kit of the disclosure contains an antiseptic agent and serves as a barrier to the growth of bacteria in the small pelvic region, thereby helping to prevent postoperative complications resulting from anastomic leakage and opening of the colorectal anastomosis.

Hydrogels belong to viscoelastic materials, whose complex rheological behavior, i.e. the behavior of substances, which include in part both viscous and elastic components, can be expressed by the so-called complex module G*. In rheology, the elastic component of deformation is expressed by the so-called elastic (memory) module (G′) and the viscous component by the so-called viscous (lossy) module (G″). Mathematically, it is possible to express a complex module as a complex number containing of a real and an imaginary component:

G*−G′+iG″

and mutual relation between G′, G″ and G* is given by the equation:

|G*|=\sqrt{G^{′2}+G^{″2}}

The process of gel formation is called gelation. In the case described, a hydrogel is formed from a polymer precursor solution which contains linear polysaccharide chains. During the chemical reaction, cross-links are formed between the individual polymer chains and thus a polymer network is formed. We call the gelation point the moment when an infinite three-dimensional network just appears in the system. The word “infinite” is to be understood as meaning that the dimensions of the resulting network are identical to the dimensions of the macroscopic gel phase. The weight fraction of the network is still insignificant at the gelation point, but in the further course it increases rapidly (the weight of the gel fraction increases at the expense of the weight of the soluble fraction), which is reflected in a gradual increase in the elastic module of the resulting hydrogel.

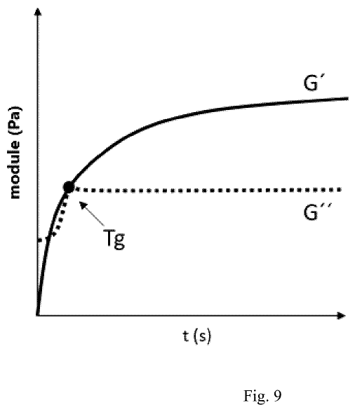

Rheologically, this change is expressed as follows: at the gelation point, the viscosity of the liquid is limited to infinity, while the module of elasticity assumes non-zero values, as can be seen from FIG. 9. At the gelation point, the elastic and viscous modules have the same value. Once the gelation point is reached, the gel formation process is not complete. As the chemical reaction continues, the polymer network increases and its rigidity increases, which is also reflected in the increase in G′. After some time, however, the crosslinking reaction stops (e.g., due to depletion of the reactants) and the polymer network stabilizes, the gel solidification process is completed. We refer to this point as solidification and the time to reach it is referred to in this text as T_{solid}. The size of the elastic module after the completion of the gel formation process is referred to herein as G's.

The object of the present disclosure is a kit for the preparation of a biodegradable hyaluronan-based hydrogel containing the antibacterial agent triclosan in the form of an inclusion with hydroxypropyl-β-cyclodextrin.

The kit as contemplated herein comprises at least two aqueous solutions A and B, one of which contains horseradish peroxidase (solution A) and the other hydrogen peroxide (solution B), whereas at least one of the solutions contains a hydroxyphenyl derivative of hyaluronan, and at least one of the precursor solutions contains triclosan in the form of an inclusion with hydroxypropyl-β-cyclodextrin. Mixing solutions A and B of the described composition in a ratio of about 1:1 forms a hydrogel based on a covalently crosslinked hydroxyphenyl derivative of hyaluronan (crossHA-TA), which its elastic module value reaches from about 100 to about 1000 Pa no later than about 3 minutes after mixing the solutions and after completion of the solidification process, its elastic module is in the range of from about 500 to about 2000 Pa. The hydrogel is formed at the site of application (in situ) and undergoes a sol-gel transition under physiological conditions. The hydrogel is intended for use during surgery, when it is able to completely fill the space of the small pelvis in the immediate vicinity of the colorectal anastomosis. The rate of solidification of the described composition allows the application of a sufficient amount of gel-forming mixture to the small pelvic area, perfect filling of the application site and the formation of a homogeneously crosslinked hydrogel in a period of time that does not excessively prolong the surgery. The resulting hydrogel provides sufficient mechanical support for the created colorectal anastomosis, but at the same time does not prevent peristaltic movements of the intestinal wall.

The use of the kit as contemplated herein allows the hydrogel used to be able to ideally fill the entire space of the small pelvis and thus surround the formed anastomosis. For this purpose, it is appropriate to use hydrogels that are able to undergo a sol-gel transition directly at the site of application under physiological conditions. The rate of the hydrogel formation process at the site of application, the homogeneity of crosslinking of the resulting hydrogel and its final viscoelastic properties are important parameters that contribute to the effectiveness of the composition. Too rapid hydrogel formation may not lead to perfect filling of the anatomically segmented area of the small pelvis and homogeneous crosslinking of the material. Inhomogeneous crosslinking can cause some of the material to leak from the application site and limit the barrier function of the device. Conversely, the slow formation of a gel can unnecessarily delay the course of surgery. Insufficient crosslinking of the hydrogel, which manifests itself as low stiffness of the hydrogel, can lead to failure of its barrier function, as it can migrate from the site of application. On the contrary, too strongly crosslinked hydrogels, showing high rigidity, can hinder the natural peristaltic movements of the intestine, and thus disrupt its function. The kit as contemplated herein thus allows the formation of a hydrogel which homogeneously fills the space of the small pelvis, surrounds the formed anastomosis and reaches a value of its elastic modulus of from about 100 to about 1000 Pa within about 3 minutes after application and its elastic module is from about 500 to about 2000 Pa after completion of the solidification process. These conditions can be met by hydrogels prepared from hydroxyphenyl hyaluronan derivatives of the general formula I as specified above, which can be crosslinked by a horseradish peroxidase catalyzed reaction, even though the presence of hydroxypropyl-β-cyclodextrin has been found to slow down the gelation rate and efficiency of the crosslinking reaction (see Example 16, FIG. 8).

To achieve the desired properties, it was therefore necessary to choose a suitable combination of the concentration of the hyaluronan derivative, its molecular weight, the degree of substitution, the concentration of hydrogen peroxide and the activity of horseradish peroxidase. The reaction is initiated by the addition of hydrogen peroxide so that the preparation of the hydrogel can be carried out by mixing two solutions of the hydroxyphenyl derivative of hyaluronan of the general formula I as specified above, where one of them contains hydrogen peroxide and the other horseradish peroxidase. Large volumes of homogeneously crosslinked hydrogel can be prepared by ensuring sufficient mixing of both precursor solutions (e.g. by using a static mixer).

Hydrogels in general may not themselves be an effective barrier against the spread of infection, as it is generally known that some types of hydrogels are used as suitable substrates for culturing bacteria. Colonization of the hydrogel by bacteria would in fact lead to a failure of the composition, as it would not prevent the development of infection in the small pelvis and its possible spread into the peritoneum. Hydrogel colonization can be prevented by combination with suitable antibacterial agents. In this case, triclosan was selected as the antimicrobial agent in the form of an inclusion with 2-hydroxypropyl-β-cyclodextrin. In vitro experiments have shown that hydrogels containing triclosan in the form of inclusions have shown in vitro antimicrobial effects and are not colonized by microorganisms.

The absorbability (biodegradability) of the material is considered to be a technically advantageous solution in the field of the development of medical devices intended for implantation into the patient's body. Even in this case, it is advantageous for the hydrogel to be absorbed after fulfilling its purpose and not to require further surgery necessary to remove it. Hydrogels based on enzymatically crosslinked hydroxyphenyl derivatives of hyaluronan as contemplated herein also show this property.

On the other hand, the hydrogel according to the present disclosure is intended to act for several days (from about 2 to about 6 days, or even more days after surgery) as a barrier to the spread of infection in the small pelvic region, outside of lumen of the digestive tract or during dehiscence of the anastomosis, exposed to the intestinal microflora. It produces a number of different types of enzymes (proteases, glycosidases-heparinases, chondroitinases, hyaluronidases), which can cause the degradation of biopolymers, including glycosaminoglycans, including hyaluronan. Premature degradation of the hydrogel at the site of application would lead to loss of its barrier function and loss of its effect. The action of enzymes of the intestinal microflora does not prevent the use of the combination with antibacterial substances, because they themselves do not prevent the person from the action of bacterial hydrolases, which can penetrate into the affected area from the damaged digestive tract. During the development of the hydrogel as contemplated herein, it has been shown that the resistance of the developed material to the action of hydrolytic enzymes (e.g. hyaluronidase) and thus its rate of degradation can be controlled by the crosslink density of the hydrogel polymer network. It was found that with increasing elastic module G′, the value of which reflects the crosslinking density of the gel, the resistance of the prepared hydrogels to the action of hydrolytic enzymes (e.g. hyaluronidase) increases and their degradation time increases. Furthermore, the presence of TCS/HP-β-CD inclusion in the hydrogel was found to further increase the resistance of hydrogels to hydrolytic enzymes (e.g. hyaluronidase) compared to hydrogels of comparable degree of crosslinking (comparable G′ and swelling coefficient Q) without TCS/HP-β-CD content. This is an advantageous property because it prolongs the time for which the hydrogel can fulfill its barrier function even in hydrogels with relatively lower G′, the consistency of which does not prevent peristaltic bowel movements.

During experiments performed on a model of dehiscence (opening or spacing) of porcine colorectal anastomosis, the presence of the above-described hydrogel of the present disclosure surrounding the collateral anastomosis and filling the small pelvis was found to prevent postoperative complications associated with development of sepsis), even if an intestinal perforation simulating partial dehiscence (disintegration) of the anastomosis was created in the vicinity of the formed anastomosis for experimental reasons. In contrast to the prior art compositions (tissue adhesives) which are applied to increase the mechanical resistance of the formed gastrointestinal tract connection, the hydrogel as contemplated herein also acts in the event of anastomosis leakage or partial anastomosis dehiscence, because in addition to its action as a support for the anastomosis and partial prevention of its mechanical damage, it also, above all, acts as a barrier preventing the escape of intestinal contents from the lumen of the colon and its spread in the area of the small pelvis. The antiseptic triclosan present prevents the colonization of the hydrogel and the small pelvic region by bacteria of the intestinal microflora, the development of peritonitis and other postoperative complications. The degree of crosslinking of the described hydrogel prevents premature degradation of the hydrogel by the action of hydrolytic enzymes produced by bacteria of the intestinal microflora but does not prevent the gradual complete absorption of the hydrogel after fulfilling its function.

The term “hyaluronan” means hyaluronic acid, or a pharmaceutically acceptable salt thereof.

### EXAMPLES

DS=degree of substitution=100%*molar amount of modified disaccharide units of hyaluronan/molar amount of all disaccharide units of the hyaluronan derivative. The degree of substitution was determined by ¹H NMR spectroscopy.

The weight average molar mass (Mw) and polydispersity index (PI) were determined by the SEC-MALLS method. Triclosan concentrations and HRP activity were determined spectrophotometrically.

### Gelation Kinetics

The gelation kinetics was determined using an AR-G2 rotary rheometer (TA instruments) using a plate-plate arrangement with a top geometry with a diameter of about 40 mm and a gap setting of about 400 μm. Precursor solutions A (about 250 μL) and B (about 250 μL) are applied onto the bottom stationary plate and pre-shear about 2000 1/s for about 1 s is used for their homogenization. The gelation kinetics is determined by the oscillation time sweep method at a frequency of about 1 Hz and a shift of about 0.001 rad at about 37° C. The gelation time is determined as the intersection of the elastic and viscous module, and the elastic module for comparing the individual samples with each other is subtracted at about 3 minutes from the start of the experiment (n=from about 3-about 5).

### Viscoelastic Properties

Hydrogels with a total volume of about 1.7±about 0.3 mL (n=from about 3-about 5) were prepared for testing and aged for about 1 hour. The viscoelastic properties of the hydrogels were determined using an AR-G2 rotary rheometer using a cross-hatch geometry with a roughened surface to prevent the prepared hydrogel from slipping. The measurement was performed in strain sweep mode at a frequency of about 1 Hz and a shift in the range of from about 0.001 to about 2 rad. For the purposes of this application, the measurement was used to determine the elastic module of the gels after solidification (G′ₛ).

### Example 1: Synthesis of Tyraminated HA Derivative (HA-TA)

Example 1A: Synthesis of 6-amino-N-[2-(4-hydroxyphenyl) ethyl]hexanamide.6[(tert-butoxycarbonyl) amino]hexanoic acid (about 1.00 g, 4.3 mmol) was dissolved in about 50 mL of tetrahydrofuran (THF). 1,1′-carbodiimidazole (about 0.70 g, 4.3 mmol) was added to the acid solution. The mixture was heated to about 50° C. for about sixty minutes. The reaction vessel was then purged with inert gas. To the reaction mixture was added tyramine (about 0.59 g, 4.3 mmol). The mixture was further heated for another about 2 hours. The THF was then removed by distillation under reduced pressure. The residue was dissolved in about 50 mL of ethyl acetate. The solution was washed with about 150 mL of purified water (divided into three parts). The organic layer was dried over a molecular sieve. The ethyl acetate was removed by distillation under reduced pressure. The residue was dissolved in about 50 mL of MeOH and about 2 mL of trifluoroacetic acid (TFA) was added to the solution. The solution was heated to reflux for about 6 hours. The solvent was removed by distillation under reduced pressure. The residue was dissolved in about 50 mL of ethyl acetate. The solution was washed with about 150 mL of purified water (divided into three parts). The organic layer was dried over a molecular sieve. The ethyl acetate was removed by distillation under reduced pressure.

m=0.75 g (70% of theory)

¹H NMR (D₂O, ppm) δ: 1.17 (m, 2H, γ-CH₂-hexanoic acid); 1.48(m, 2 H, β-CH₂-hexanoic acid); 1.58 (m, 2H, δ-CH₂-hexanoic acid); 2.17 (t, 2H, —CH₂—CO—); 2.73 (m, 2H, —CH₂-Ph); 2.91 (m, 2H, —CH₂—NH₂); 3.42 (m, 2H, —CH₂—NH—CO—); 6.83 (d, 2H, arom); 7.13 (d, 2H, arom).

¹³C NMR (D₂O, ppm) δ: 24 (γ-C-hexanoic acid); 26 (β-C-hexanoic acid); 33 (0-C-hexanoic acid); 35 (—C—CO—); 39 (—C—NH₂); 40 (C-Ph); 63 (—C—NH—CO—); 115 (C3 arom); 126 (C1 arom); 130 (C2 arom.); 153 (C4 arom); 176 (—CO—).

### Example 1B: Preparation of an Aldehyde Derivative (HA-CHO)

Hylauronan (10.00 g, M, =2,000,000 g/mol) was dissolved in about 750 mL of a 2.5% (w/w) Na₂HPO₄·12 H₂O solution. The solution was cooled to about 5° C. To the resulting solution were added about 2.60 g of NaBr and about 0.05 g of 4-acetamido-2,2,6,6-tetramethylpiperidine-1-oxyl. After thorough homogenization of the solution, about 3 mL of NaClO solution (from about 10-about 15% of available C12) were added to the reaction mixture. The reaction was continued with stirring for about 15 min. The reaction was quenched by the addition of about 100 mL of about 40% propan-2-ol solution. The product was purified by ultrafiltration and isolated by precipitation with propan-2-ol.

IR (KBr): 3417, 2886, 2152, 1659, 1620, 1550, 1412, 1378, 1323, 1236, 1204, 1154, 1078, 1038, 945, 893 cm⁻¹.

¹H NMR (D₂O) δ: 2.01 (s, 3H, CH₃—), 3.37-3.93 (m, skeleton of hyaluronan), 4.46 (s, 1H, anomer), 4.54 (s, 1H anomer, —O—CH(OH)—), 5.27 (geminal glycol —CH—(OH)₂).

### Example 1C: Preparation of a Tyraminated HA Derivative with a C6 Spacer (Mw≈ 80,000 g/mol, DS≈ 3%)

The aldehyde derivative of HA (˜ 60,000 g/mol, DS=9%) (5.00 g) was dissolved in about 500 mL of demineralized water. The pH of the solution was adjusted to about 3 with acetic acid. To a solution of HA-CHO was added 6-amino-N-[2-(4-hydroxyphenyl) ethyl]hexanamide (intermediate (I)) (about 1.25 g, 5 mmol). The mixture was stirred at room temperature for about 2 hours. Then picoline-borane complex (about 0.270 g, 2.5 mmol) was added to the reaction mixture. The mixture was stirred for another about 12 hours at room temperature. The product was purified by ultrafiltration and isolated from the retentate by propan-2-ol precipitation. The precipitate was freed of moisture and residual propan-2-ol by drying in a hot air oven (about 40° C., about 3 days).

IR (KBr): 3425, 2893, 2148, 1660, 1620, 1549, 1412, 1378, 1323, 1236, 1204, 1154, 1078, 1038, 945, 893 cm⁻¹.

¹H NMR (D₂O) δ: 1.25 (t, 2H, γ-CH₂— aminohexane acids), 1.48 (m, 2H, δ-CH₂-aminohexane acids), 1.51 (m, 2H, β-CH₂— aminohexane acids), 2.01 (s, 3H, CH₃—), 2.65 (m, 2H, Ph-CH₂—), 2.73 (m, 2H, ϵ—CH₂— aminohexane acids), 3.37-3.93 (m, skeleton of hyaluronan), 4.46 (s, 1H, anomer), 4.54 (s, 1H anomer, —O—CH(OH)—), 6.59 (d, 2H, arom.), 7.01 (d, 2H, arom).

SEC MALLS: Mw=83,000 g/mol; PI=1.61

DS (¹H NMR): 2.7%

### Example 1D: Preparation of a Tyraminated HA Derivative with a C6 Spacer (Mw≈300,000 g/mol, DS≈2%)

The aldehyde derivative of HA (Mw≈300,000 g/mol, DS≈9%) (5.00 g) was dissolved in about 500 mL of demineralized water. The pH of the solution was adjusted to about 3 with acetic acid. To a solution of HA-CHO was added 6-amino-N-[2-(4-hydroxyphenyl) ethyl]hexanamide (intermediate (I)) (about 0.625 g, 2.5 mmol). The mixture was stirred at room temperature for about 2 hours. Then picoline-borane complex (about 0.270 g, 2.5 mmol) was added to the reaction mixture. The mixture was stirred for another about 12 hours at room temperature. The product was purified by ultrafiltration and isolated from the retentate by propan-2-ol precipitation. The precipitate was freed of moisture and residual propan-2-ol by drying in a hot air oven (about 40° C., about 3 days).

IR (KBr): 3425, 2893, 2148, 1660, 1620, 1549, 1412, 1378, 1323, 1236, 1204, 1154, 1078, 1038, 945, 893 cm⁻¹.

¹H NMR (D₂O) δ: 1.25 (t, 2H, γ-CH₂— aminohexane acids), 1.48 (m, 2H, δ-CH₂-aminohexane acids), 1.51 (m, 2H, β-CH₂— aminohexane acids), 2.01 (s, 3H, CH₃—), 2.65 (m, 2H, Ph-CH₂—), 2.73 (m, 2H, ϵ—CH₂— aminohexane acids), 3.37-3.93 (m, skeleton of hyaluronan), 4.46 (s, 1H, anomer), 4.54 (s, 1H anomer., —O—CH(OH)—), 6.59 (d, 2H, arom.), 7.01 (d, 2H, arom).

SEC MALLS: Mw=281,000 g/mol; PI=1.49

DS (¹H NMR): 2.3%

### Example 1E: Preparation of Tyraminated Derivative of HA with C6 Spacer (Mw≈1,000,000 g/mol, DS≈1%)

The aldehyde derivative of HA (≈1,500,000 g/mol, DS=4%) (5.00 g) was dissolved in about 500 mL demineralized water. The pH of the solution was adjusted to about 3 with acetic acid. To a solution of HA-CHO was added 6-amino-N-[2-(4-hydroxyphenyl) ethyl]hexanamide (intermediate (I)) (about 0.625 g, 2.5 mmol). The mixture was stirred at room temperature for about 2 hours. Then picoline-borane complex (about 0.270 g, 2.5 mmol) was added to the reaction mixture. The mixture was stirred for another about 12 hours at room temperature. The product was purified by ultrafiltration and isolated from the retentate by propan-2-ol precipitation. The precipitate was freed of moisture and residual propan-2-ol by drying in a hot air oven (about 40° C., about 3 days).

IR (KBr): 3425, 2893, 2148, 1660, 1620, 1549, 1412, 1378, 1323, 1236, 1204, 1154, 1078, 1038, 945, 893 cm⁻¹.

¹H NMR (D₂O) δ: 1.25 (t, 2H, γ-CH₂— aminohexane acids), 1.48 (m, 2H, δ-CH₂-aminohexane acids), 1.51 (m, 2H, β-CH₂— aminohexane acids), 2.01 (s, 3H, CH₃—), 2.65 (m, 2H, Ph-CH₂—), 2.73 (m, 2H, ϵ—CH₂— aminohexane acids), 3.37-3.93 (m, skeleton of hyaluronanu), 4.46 (s, 1H, anomer), 4.54 (s, 1H anomer., —O—CH(OH)—), 6.59 (d, 2H, arom.), 7.01 (d, 2H. arom).

SEC MALLS: Mw=1,000,000 g/mol; PI=1.65

DS (¹H NMR): 0.9%

### Example 1F: Preparation of a Tyraminated HA Derivative with a C6 Spacer (Mw≈2,000,000 g/mol DS z 1%)

The aldehyde derivative of HA (≈100,000 g/mol, DS=10%) (5.00 g) was dissolved in about 500 mL of demineralized water. The pH of the solution was adjusted to about 3 with acetic acid. To a solution of HA-CHO was added 6-amino-N-[2-(4-hydroxyphenyl) ethyl]hexanamide (intermediate (I)) about (0.625 g, 2.5 mmol). The mixture was stirred at room temperature for about 2 hours. Then picoline-borane complex (about 0.270 g, 2.5 mmol) was added to the reaction mixture. The mixture was stirred for another about 12 hours at room temperature. The product was purified by ultrafiltration and isolated from the retentate by propan-2-ol precipitation. The precipitate was freed of moisture and residual propan-2-ol by drying in a hot air oven (about 40° C., about 3 days).

IR (KBr): 3425, 2893, 2148, 1660, 1620, 1549, 1412, 1378, 1323, 1236, 1204, 1154, 1078, 1038, 945, 893 cm⁻¹.

¹H NMR (D₂O) δ: 1.25 (t, 2H, γ-CH₂— aminohexane acids), 1.48 (m, 2H, δ-CH₂-aminohexane acids), 1.51 (m, 2H, β-CH₂— aminohexane acids), 2.01 (s, 3H, CH₃—), 2.65 (m, 2H, Ph-CH₂—), 2.73 (m, 2H, ϵ—CH₂— aminohexane acids), 3.37-3.93 (m, skeleton of hyaluronan), 4.46 (s, 1H, anomer), 4.54 (s, 1H anomer., —O—CH(OH)—), 6.59 (d, 2H, arom.), 7.01 (d, 2H. arom).

SEC MALLS: Mw=1,800,000 kDa; PI=1.55

DS (¹H NMR): 1.0%

### Example 1G: Preparation of a Tyraminated HA Derivative with a C6 Spacer (Mw≈700,000 g/mol, DS=3%)

The aldehyde derivative of HA (≈2,500,000 g/mol, DS=4%) (5.00 g) was dissolved in about 500 mL demineralized water. The pH of the solution was adjusted to about 3 with acetic acid. To a solution of HA-CHO was added 6-amino-N-[2-(4-hydroxyphenyl) ethyl]hexanamide (intermediate (I)) (about 0.625 g, 2.5 mmol). The mixture was stirred at room temperature for about 2 hours. Then picoline-borane complex (about 0.270 g, 2.5 mmol) was added to the reaction mixture. The mixture was stirred for another about 12 hours at room temperature. The product was purified by ultrafiltration and isolated from the retentate by propan-2-ol precipitation. The precipitate was freed of moisture and residual propan-2-ol by drying in a hot air oven (about 40° C., about 3 days).

IR (KBr): 3425, 2893, 2148, 1660, 1620, 1549, 1412, 1378, 1323, 1236, 1204, 1154, 1078, 1038, 945, 893 cm⁻¹.

¹H NMR (D₂O) δ: 1.25 (t, 2H, γ-CH₂— aminohexane acids), 1.48 (m, 2H, δ-CH₂-aminohexane acids), 1.51 (m, 2H, β-CH₂— aminohexane acids), 2.01 (s, 3H, CH₃—), 2.65 (m, 2H, Ph-CH₂—), 2.73 (m, 2H, ϵ—CH₂— aminohexane acids), 3.37-3.93 (m, skeleton of hyaluronan), 4.46 (s, 1H, anomer), 4.54 (s, 1H anomer., —O—CH(OH)—), 6.59 (d, 2H, arom.), 7.01 (d, 2H. arom).

SEC MALLS: Mw=700,000 g/mol; PI=1.65

DS (¹H NMR): 3.2%

### Example 11: Preparation of a Tyraminated HA Derivative with a C6 Spacer (Mw≈90,000 g/mol, DS=7%)

The aldehyde derivative of HA (≈2,500,000 g/mol, DS=4%) (5.00 g) was dissolved in about 500 mL demineralized water. The pH of the solution was adjusted to about 3 with acetic acid. To a solution of HA-CHO was added 6-amino-N-[2-(4-hydroxyphenyl) ethyl]hexanamide (intermediate (I)) (about 0.625 g, 2.5 mmol). The mixture was stirred at room temperature for about 2 hours. Then picoline-borane complex (about 0.270 g, 2.5 mmol) was added to the reaction mixture. The mixture was stirred for another about 12 hours at room temperature. The product was purified by ultrafiltration and isolated from the retentate by propan-2-ol precipitation. The precipitate was freed of moisture and residual propan-2-ol by drying in a hot air oven (about 40° C., about 3 days).

IR (KBr): 3425, 2893, 2148, 1660, 1620, 1549, 1412, 1378, 1323, 1236, 1204, 1154, 1078, 1038, 945, 893 cm⁻¹.

¹H NMR (D₂O) δ: 1.25 (t, 2H, γ-CH₂— aminohexane acids), 1.48 (m, 2H, δ-CH₂-aminohexane acids), 1.51 (m, 2H, β-CH₂— aminohexane acids), 2.01 (s, 3H, CH₃—), 2.65 (m, 2H, Ph-CH₂—), 2.73 (m, 2H, ϵ—CH₂— aminohexane acids), 3.37-3.93 (m, skeleton of hyaluronan), 4.46 (s, 1H, anomer), 4.54 (s, 1H anomer., —O—CH(OH)—), 6.59 (d, 2H, arom.), 7.01 (d, 2H. arom).

SEC MALLS: Mw=91,000 g/mol; PI=1.65

DS (¹H NMR): 7.2%

### Example 2: Preparation of a Hydrogel by Mixing Solutions A and B

A HA-TA derivative prepared according to the procedure of Example 1D was used to prepare solutions of the features for hydrogel preparation. The concentrations of the individual components of the solution A and the solution B are given in table 1.

TABLE 1
Composition of precursor solutions
A and B for hydrogel preparation

| Solution A | | | Solution B | | |
|---|---|---|---|---|---|
| HRP | 0.846 | U/mL | H₂O₂ | 3.3 | mmol/L |
| HA-TA | 20 | mg/mL | HA-TA | 20 | mg/mL |
| Hydroxypropyl-β-cyklodextrin | 60 | mg/mL | | | |
| Triclosan | 1.44 | mg/mL | | | |

The hydrogel was prepared by mixing solutions A and B in a ratio of about 1:1. The hydrogel thus prepared contains the enzyme horseradish peroxidase, a covalently crosslinked hydroxyphenyl derivative of hyaluronan (crossHA-TA), hydroxypropyl-β-cyclodextrin and triclosan. The final composition of the hydrogel, including the values of the gelation time (Tg) and the value of the elastic module G′ after about 3 min (G′_{3 min}) and after the completion of solidification (G'ₛ) are given in Table 2.

TABLE 2
Composition and parameters of the prepared hydrogel

| | |
|---|---|
| HRP (U/mL) | 0.423 |
| crossHA-TA (mg/mL) | 20 |
| Hydroxypropyl-β-cyclodextrin (mg/mL) | 30 |
| Triclosan (mg/mL) | 0.72 |
| Tg (s) | 65 |
| G′_{3 min} (Pa) | 105 |
| G′ₛ (Pa) | 591 |
| ratio TCS:HP-β-CD (mol/mol) | 1:9 |

### Example 3: Preparation of a Hydrogel by Mixing Solutions A and B

The HA-derivative prepared according to Example 1D was used to prepare solutions of the features for the hydrogel preparation. The concentrations of the individual components of the solution A and the solution B are given in Table 3

TABLE 3
Composition of precursor solutions
A and B for hydrogel preparation

| Solution A | | | Solution B | | |
|---|---|---|---|---|---|
| HRP | 1.2 | U/mL | H₂O₂ | 4.66 | mmol/L |
| HA-TA | 20 | mg/mL | HA-TA | 20 | mg/mL |
| Hydroxypropyl-β-cyclodextrin | 60 | mg/mL | | | |
| Triclosan | 2.04 | mg/mL | | | |

The hydrogel was prepared by mixing solutions A and B in a ratio of about 1:1. The hydrogel thus prepared contains the enzyme horseradish peroxidase, a covalently crosslinked hydroxyphenyl derivative of hyaluronan (crossHA-TA), hydroxypropyl-β-cyclodextrin and triclosan. The final composition of the hydrogel, including the values of the gelation time (Tg) and the value of the elastic module G′ after about 3 min (G′_{3 min}) and after the completion of solidification (G′ₛ) are given in Table 4.

TABLE 4
Composition and parameters of the prepared hydrogel

| | |
|---|---|
| HRP (U/mL) | 0.6 |
| crossHA-TA (mg/mL) | 20 |
| Hydroxypropyl-β-cyklodextrin (mg/mL) | 30 |
| Triclosan (mg/mL) | 1.02 |
| Tg (s) | 28 |
| G′_{3 min} (Pa) | 440 |
| G′ₛ (Pa) | 1118 |
| ratio TCS:HP-β-CD (mol/mol) | 1:6 |

### Example 4: Preparation of a Hydrogel by Mixing Solutions A and B

The HA-TA derivative (prepared according to Example 1D) was used to prepare solutions of the features for hydrogel preparation. Concentration of individual components of the solution A and the solution B are given in Table 5.

TABLE 5
Composition of precursor solutions
A and B for hydrogel preparation

| Solution A | | | Solution B | | |
|---|---|---|---|---|---|
| HRP | 0.835 | U/mL | H₂O₂ | 4.66 | mmol/L |
| HA-TA | 20 | mg/mL | HA-TA | 20 | mg/mL |
| Hydroxypropyl-β-cyclodextrin | 60 | mg/mL | | | |
| Triclosan | 2.0 | mg/mL | | | |

The hydrogel was prepared by mixing solutions A and B in a ratio of about 1:1. The hydrogel thus prepared contains the enzyme horseradish peroxidase, a covalently crosslinked hydroxyphenyl derivative of hyaluronan (crossHA-TA), hydroxypropyl-β-cyclodextrin and triclosan. The final composition of the hydrogel, including the values of the gelation time (Tg) and the value of the elastic module G′ after about 3 min (G′_{3 min}) and after the completion of solidification (G′ₛ) are given in Table 6.

TABLE 6
Composition and parameters of the prepared hydrogel

| | |
|---|---|
| HRP (U/mL) | 0.418 |
| crossHA-TA (mg/mL) | 20 |
| Hydroxypropyl-β-cyclodextrin (mg/mL) | 30 |
| Triclosan (mg/mL) | 1.0 |
| Tg (s) | 38 |
| G′_{3 min} (Pa) | 290 |
| G′ₛ (Pa) | 1051 |
| ratio TCS:HP-β-CD (mol/mol) | 1:6 |

### Example 5: Preparation of a Hydrogel by Mixing Solutions A and B

The HA-TA derivative prepared according to Example 1D was used to prepare solutions of the features for the hydrogel preparation. The concentrations of the individual components of the solution A and the solution B are given in Table 7.

TABLE 7
Composition of precursor solutions
A and B for hydrogel preparation

| Solution A | | | Solution B | | |
|---|---|---|---|---|---|
| HRP | 1.08 | U/mL | H₂O₂ | 4.66 | mmol/L |
| HA-TA | 20 | mg/mL | HA-TA | 20 | mg/mL |
| Hydroxypropyl-β-cyclodextrin | 60 | mg/mL | | | |
| Triclosan | 2.02 | mg/mL | | | |

The hydrogel was prepared by mixing solutions A and B in a ratio of about 1:1. The hydrogel thus prepared contains the enzyme horseradish peroxidase, a covalently crosslinked hydroxyphenyl derivative of hyaluronan (crossHA-TA), hydroxypropyl-β-cyclodextrin and triclosan. The final composition of the hydrogel, including the values of the gelation time (Tg) and the value of the elastic module G′ after about 3 min (G′_{3 min}) and after the completion of solidification (G′ₛ) are given in Table 8.

TABLE 8
Composition and parameters of the prepared hydrogel

| | |
|---|---|
| HRP (U/mL) | 0.54 |
| crossHA-TA (mg/mL) | 20 |
| Hydroxypropyl-β-cyclodextrin (mg/mL) | 30 |
| Triclosan (mg/mL) | 1.01 |
| Tg (s) | 28 |
| G′_{3 min} (Pa) | 430 |
| G′ₛ (Pa) | 1122 |
| ratio TCS:HP-β-CD (mol/mol) | 1:6 |

### Example 6: Preparation of a Hydrogel by Mixing Solutions A and B

The HA-TA derivative prepared according to Example 1D was used to prepare solutions of the features for the hydrogel preparation. The concentrations of the individual components of the solution A and the solution B are given in Table 9.

TABLE 9
Composition of precursor solutions
A and B for hydrogel preparation

| Solution A | | | Solution B | | |
|---|---|---|---|---|---|
| HRP | 1.17 | U/mL | H₂O₂ | 4.66 | mmol/L |
| HA-TA | 20 | mg/mL | HA-TA | 20 | mg/mL |
| Hydroxypropyl-β-cyclodextrin | 60 | mg/mL | | | |
| Triclosan | 1.88 | mg/mL | | | |

The hydrogel was prepared by mixing solutions A and B in a ratio of about 1:1. The hydrogel thus prepared contains the enzyme horseradish peroxidase, a covalently crosslinked hydroxyphenyl derivative of hyaluronan (crossHA-TA), hydroxypropyl-β-cyclodextrin and triclosan. The final composition of the hydrogel, including the values of the gelation time (Tg) and the value of the elastic module G′ after about 3 min (G′_{3 min}) and after the completion of solidification (G′ₛ) are given in Table 10.

TABLE 10
Composition and parameters of the prepared hydrogel

| | |
|---|---|
| HRP (U/mL) | 0.59 |
| crossHA-TA (mg/mL) | 20 |
| Hydroxypropyl-β-cyklodextrin (mg/mL) | 30 |
| Triclosan (mg/mL) | 0.94 |
| Tg (s) | 31 |
| G′_{3 min} (Pa) | 394 |
| G′ₛ (Pa) | 1126 |
| ratio TCS:HP-β-CD (mol/mol) | 1:7 |

### Example 7: Preparation of a Hydrogel by Mixing Solutions A and B

The HA-TA derivative prepared according to Example 1D was used to prepare solutions of the features for the hydrogel preparation. The concentrations of the individual components of the solution A and the solution B are given in Table 11.

TABLE 11
Composition of precursor solutions
A and B for hydrogel preparation

| Solution A | | | Solution B | | |
|---|---|---|---|---|---|
| HRP | 1.31 | U/mL | H₂O₂ | 4.66 | mmol/L |
| HA-TA | 20 | mg/mL | HA-TA | 20 | mg/mL |
| Hydroxypropyl-β-cyclodextrin | 60 | mg/mL | | | |
| Triclosan | 1.87 | mg/mL | | | |

The hydrogel was prepared by mixing solutions A and B in a ratio of about 1:1. The hydrogel thus prepared contains the enzyme horseradish peroxidase, a covalently crosslinked hydroxyphenyl derivative of hyaluronan (crossHA-TA), hydroxypropyl-β-cyclodextrin and triclosan. The final composition of the hydrogel, including the values of the gelation time (Tg) and the value of the elastic module G′ after about 3 min (G′_{3 min}) and after the completion of solidification (G′ₛ) are given in Table 12.

TABLE 12
Composition and parameters of the prepared hydrogel

| | |
|---|---|
| HRP (U/mL) | 0.66 |
| crossHA-TA (mg/mL) | 20 |
| Hydroxypropyl-β-cyklodextrin (mg/mL) | 30 |
| Triclosan (mg/mL) | 0.94 |
| Tg (s) | 30 |
| G′_{3 min} (Pa) | 401 |
| G′ₛ (Pa) | 1127 |
| ratio TCS:HP-β-CD (mol/mol) | 1:7 |

### Example 8: Preparation of a Hydrogel by Mixing Solutions A and B

The HA-TA derivative prepared according to Example 1D was used to prepare solutions of the features for the hydrogel preparation. The concentrations of the individual components of the solution A and the solution B are given in Table 13.

TABLE 13
Composition of precursor solutions
A and B for hydrogel preparation

| Solution A | | | Solution B | | |
|---|---|---|---|---|---|
| HRP | 1.13 | U/mL | H₂O₂ | 4.66 | mmol/L |
| HA-TA | 20 | mg/mL | HA-TA | 20 | mg/mL |
| Hydroxypropyl-β-cyclodextrin | 60 | mg/mL | | | |
| Triclosan | 1.86 | mg/mL | | | |

The hydrogel was prepared by mixing solutions A and B in a ratio of about 1:1. The hydrogel thus prepared contains the enzyme horseradish peroxidase, a covalently crosslinked hydroxyphenyl derivative of hyaluronan (crossHA-TA), hydroxypropyl-β-cyclodextrin and triclosan. The final composition of the hydrogel, including the values of the gelation time (Tg) and the value of the elastic module G′ after about 3 min (G′_{3 min}) and after the completion of solidification (G′ₛ) are given in Table 14.

TABLE 14
Composition and parameters of the prepared hydrogel

| | |
|---|---|
| HRP (U/mL) | 0.56 |
| crossHA-TA (mg/mL) | 20 |
| Hydroxypropyl-β-cyklodextrin (mg/mL) | 30 |
| Triclosan (mg/mL) | 0.93 |
| Tg (s) | 36 |
| G′_{3 min} (Pa) | 293 |
| G′ₛ (Pa) | 1055 |
| ratio TCS:HP-β-CD (mol/mol) | 1:7 |

### Example 9: Preparation of a Hydrogel by Mixing Solutions A and B

The HA-TA derivative prepared according to Example 1D was used to prepare solutions of the features for the hydrogel preparation. The concentrations of the individual components of the solution A and the solution B are given in Table 15.

TABLE 15
Composition of precursor solutions
A and B for hydrogel preparation

| | |
|---|---|
| Solution A | Solution B |

| | | | | | |
|---|---|---|---|---|---|
| HRP | 1.12 | U/mL | H₂O₂ | 4.66 | mmol/L |
| HA-TA | 20 | mg/mL | HA-TA | 20 | mg/mL |
| Hydroxypropyl-β- | 60 | mg/mL | | | |
| cyklodextrin | | | | | |
| Triclosan | 2.08 | mg/mL | | | |

The hydrogel was prepared by mixing solutions A and B in a ratio of about 1:1. The hydrogel thus prepared contains the enzyme horseradish peroxidase, a covalently crosslinked hydroxyphenyl derivative of hyaluronan (crossHA-TA), hydroxypropyl-β-cyclodextrin and triclosan. The final composition of the hydrogel, including the values of the gelation time (Tg) and the value of the elastic module G′ after about 3 min (G′_{3 min}) and after the completion of solidification (G′ₛ) are given in Table 16.

TABLE 16
Composition and parameters of the prepared hydrogel

| | |
|---|---|
| HRP (U/mL) | 0.56 |
| crossHA-TA (mg/mL) | 20 |
| Hydroxypropyl-β-cyklodextrin (mg/mL) | 30 |
| Triclosan (mg/mL) | 1.04 |
| Tg (s) | 28 |
| G′_{3 min} (Pa) | 349 |
| G′ₛ (Pa) | 1110 |
| ratio TCS:HP-β-CD | 1:6 |

### Example 10: Preparation of a Hydrogel by Mixing Solutions A and B

The HA-TA derivative prepared according to Example 1D was used to prepare solutions of the features for the hydrogel preparation. The concentrations of the individual components of solution A and B are given in Table 17.

TABLE 17
Composition of precursor solutions
A and B for hydrogel preparation

| | |
|---|---|
| Solution A | Solution B |

| | | | | | |
|---|---|---|---|---|---|
| HRP | 0.52 | U/mL | H₂O₂ | 1.33 | mmol/L |
| HA-TA | 20 | mg/mL | HA-TA | 20 | mg/mL |
| Hydroxypropyl-β- | 4 | mg/mL | | | |
| cyklodextrin | | | | | |
| Triclosan | 0.20 | mg/mL | | | |

The hydrogel was prepared by mixing solutions A and B in a ratio of about 1:1. The hydrogel thus prepared contains the enzyme horseradish peroxidase, a covalently crosslinked hydroxyphenyl derivative of hyaluronan (crossHA-TA), hydroxypropyl-β-cyclodextrin and triclosan. The final composition of the hydrogel, including the values of the gelation time (Tg) and the value of the elastic module G′ after about 3 min (G′_{3 min}) and after the completion of solidification (G′ₛ) are given in Table 18.

TABLE 18
Composition and parameters of the prepared hydrogel

| | |
|---|---|
| HRP (U/mL) | 0.26 |
| crossHA-TA (mg/mL) | 20 |
| Hydroxypropyl-β-cyklodextrin (mg/mL) | 2 |
| Triclosan (mg/mL) | 0.10 |
| Tg (s) | 50 |
| G′_{3 min} (Pa) | 488 |
| G′ₛ (Pa) | 1081 |
| atio TCS:HP-β-CD (mol/mol) | 1:4 |

### Example 11: Preparation of a Hydrogel by Mixing Solutions A and B

The HA-TA derivative prepared according to Example 1F was used to prepare solutions of the features for the hydrogel preparation. The concentrations of the individual components of the solution A and the solution B are given in Table 19.

TABLE 19
Composition of precursor solutions
A and B for hydrogel preparation

| Solution A | | | Solution B | | |
|---|---|---|---|---|---|
| HRP | 0.72 | U/mL | H₂O₂ | 2.33 | mmol/L |
| HA-TA | 10 | mg/mL | HA-TA | 10 | mg/mL |
| Hydroxypropyl-β-cyclodextrin | 6 | mg/mL | | | |
| Triclosan | 0.20 | mg/mL | | | |

The hydrogel was prepared by mixing solutions A and B in a ratio of about 1:1. The hydrogel thus prepared contains the enzyme horseradish peroxidase, a covalently crosslinked hydroxyphenyl derivative of hyaluronan (crossHA-TA), hydroxypropyl-β-cyclodextrin and triclosan. The final composition of the hydrogel, including the values of the gelation time (Tg) and the value of the elastic module G′ after about 3 min (G′_{3 min}) and after the completion of solidification (G′ₛ) are given in Table 20.

TABLE 20
Composition and parameters of the prepared hydrogel

| | |
|---|---|
| HRP (U/mL) | 0.36 |
| crossHA-TA (mg/mL) | 10 |
| Hydroxypropyl-β-cyklodextrin (mg/mL) | 8 |
| Triclosan (mg/mL) | 0.10 |
| Tg (s) | 43 |
| G′_{3 min} (Pa) | 506 |
| G′ₛ (Pa) | 1060 |
| ratio TCS:HP-β-CD (mol/mol) | 1:6 |

### Example 12: Preparation of a Hydrogel by Mixing Solutions A and B

The HA-TA derivative prepared according to Example 1E was used to prepare solutions of the features for the hydrogel preparation. The concentrations of the individual components of the solution A and the solution B are given in Table 21

TABLE 21
Composition of precursor solutions
A and B for hydrogel preparation

| Solution A | | | Solution B | | |
|---|---|---|---|---|---|
| HRP | 0.72 | U/mL | H₂O₂ | 2.33 | mmol/L |
| HA-TA | 10 | mg/mL | HA-TA | 10 | mg/mL |
| Hydroxypropyl-β- | 8 | mg/mL | Hydroxypropyl-β- | 8 | mg/mL |
| cyclodextrin | | | cyclodextrin | | |
| Triclosan | 0.20 | mg/mL | Triclosan | 0.20 | mg/mL |

The hydrogel was prepared by mixing solutions A and B in a ratio of about 1:1. The hydrogel thus prepared contains the enzyme horseradish peroxidase, a covalently crosslinked hydroxyphenyl derivative of hyaluronan (crossHA-TA), hydroxypropyl-β-cyclodextrin and triclosan. The final composition of the hydrogel, including the values of the gelation time (Tg) and the value of the elastic module G′ after about 3 min (G′_{3 min}) and after the completion of solidification (G′ₛ) are given in Table 22.

TABLE 22
Composition and parameters of the prepared hydrogel

| | |
|---|---|
| HRP (U/mL) | 0.36 |
| crossHA-TA (mg/mL) | 10 |
| Hydroxypropyl-β-cyclodextrin (mg/mL) | 8 |
| Triclosan (mg/mL) | 0.20 |
| Tg (s) | 35 |
| G′_{3 min} (Pa) | 549 |
| G′ₛ (Pa) | 994 |
| ratio TCS:HP-β-CD (mol/mol) | 1:8 |

### Example 13: Preparation of a Hydrogel by Mixing Solutions A and B

The HA-derivative prepared according to Example 1G was used to prepare solutions of the features for the hydrogel preparation. The concentrations of the individual components of the solution A and the solution B are given in Table 23.

TABLE 23
Composition of precursor solutions
A and B for hydrogel preparation

| Solution A | | | Solution B | | |
|---|---|---|---|---|---|
| HRP | 1.12 | U/mL | H₂O₂ | 4.66 | mmol/L |
| HA-TA | 20 | mg/mL | HA-TA | 20 | mg/mL |
| Hydroxypropyl-β- | 30 | mg/mL | Hydroxypropyl-β- | 30 | mg/mL |
| cyclodextrin | | | cyclodextrin | | |
| Triclosan | 1 | mg/mL | Triclosan | 1 | mg/mL |

The hydrogel was prepared by mixing solutions A and B in a ratio of about 1:1. The hydrogel thus prepared contains the enzyme horseradish peroxidase, a covalently crosslinked hydroxyphenyl derivative of hyaluronan (crossHA-TA), hydroxypropyl-β-cyclodextrin and triclosan. The final composition of the hydrogel, including the values of the gelation time (Tg) and the value of the elastic module G′ after about 3 min (G′_{3 min}) and after the completion of solidification (G′ₛ) are given in Table 24.

TABLE 24
Composition and parameters of the prepared hydrogel

| | |
|---|---|
| HRP (U/mL) | 0.56 |
| crossHA-TA (mg/mL) | 20 |
| Hydroxypropyl-β-cyclodextrin (mg/mL) | 30 |
| Triclosan (mg/mL) | 1 |
| Tg (s) | 16 |
| G′_{3 min} (Pa) | 588 |
| G′ₛ (Pa) | 1850 |
| ratio TCS:HP-β-CD (mol/mol) | 1:6 |

### Example 14: Preparation of a Hydrogel by Mixing Solutions A and B

The HA-TA derivative (prepared according to Example 11) was used to prepare solutions of the features for hydrogel preparation. The concentrations of the individual components of the solution A and the solution B are given in Table 25.

TABLE 25
Composition of precursor solutions
A and B for hydrogel preparation

| Solution A | | | Solution B | | |
|---|---|---|---|---|---|
| HRP | 1.12 | U/mL | H₂O₂ | 2.33 | mmol/L |
| HA-TA | 40 | mg/mL | HA-TA | 40 | mg/mL |
| | | | Hydroxypropyl-β-cyclodextrin | 40 | mg/mL |
| | | | Triclosan | 2 | mg/mL |

The hydrogel was prepared by mixing solutions A and B in a ratio of about 1:1. The hydrogel thus prepared contains the enzyme horseradish peroxidase, a covalently crosslinked hydroxyphenyl derivative of hyaluronan (crossHA-TA), hydroxypropyl-β-cyclodextrin and triclosan. The final composition of the hydrogel, including the values of the gelation time (Tg) and the value of the elastic module G′ after about 3 min (G′_{3 min}) and after the completion of solidification (G′ₛ) are given in Table 26.

TABLE 26
Composition and parameters of the prepared hydrogel

| | |
|---|---|
| HRP (U/mL) | 0.6 |
| crossHA-TA (mg/mL) | 40 |
| Hydroxypropyl-β-cyklodextrin (mg/mL) | 20 |
| Triclosan (mg/mL) | 1 |
| Tg (s) | 37 |
| G′_{3 min} (Pa) | 830 |
| G′ₛ (Pa) | 1530 |
| ratio TCS:HP-β-CD (mol/mol) | 1:4 |

### Example 15: Preparation of a Hydrogel by Mixing Solutions A and B

The HA-TA derivative prepared according to Example 1C was used to prepare solutions of the features for the hydrogel preparation. The concentrations of the individual components of the solution A and the solution B are given in Table 27.

TABLE 27
Composition of precursor solutions
A and B for hydrogel preparation

| Solution A | | | Solution B | | |
|---|---|---|---|---|---|
| HRP | 1.12 | U/mL | H₂O₂ | 2 | mmol/L |
| HA-TA | 20 | mg/mL | HA-TA | 20 | mg/mL |
| | | | Hydroxypropyl-β-cyclodextrin | 80 | mg/mL |
| | | | Triclosan | 2 | mg/mL |

The hydrogel was prepared by mixing solutions A and B in a ratio of about 1:1. The hydrogel thus prepared contains the enzyme horseradish peroxidase, a covalently crosslinked hydroxyphenyl derivative of hyaluronan (crossHA-TA), hydroxypropyl-β-cyclodextrin and triclosan. The final composition of the hydrogel, including the values of the gelation time (Tg) and the value of the elastic modulus G′ after about 3 min (G′_{3 min}) and after the completion of solidification (G′ₛ) are given in Table 28.

TABLE 28
Composition and parameters of the prepared hydrogel

| | |
|---|---|
| HRP (U/mL) | 0.6 |
| crossHA-TA (mg/mL) | 20 |
| Hydroxypropyl-β-cyklodextrin (mg/mL) | 40 |
| Triclosan (mg/mL) | 1 |
| Tg (s) | 35 |
| G′_{3 min} (Pa) | 387 |
| G′ₛ (Pa) | 850 |
| ratio TCS:HP-β-CD (mol/mol) | 1:8 |

### Example 16: Influence of Hydrogel Crosslink Density and Presence of TCS/HP-β-CD Inclusion on the Rate of its Degradation by Hyaluronidase In Vitro

Three types of hydrogels without inclusion of TCS/HP-β-CD (Type A, B, C) were prepared, which differed in their viscoelastic properties and swelling coefficient. A hydroxyphenyl derivative of hyaluronan with a Mw of about 281,000 g/mol and a DS of about 2.2% was used for preparation of the hydrogels. The concentrations of the reagents used for the preparation of hydrogels are given in Table 29. The crosslinking density and thus the viscoelastic properties of the hydrogels were regulated by the concentration of crosslinking agents—HRP and H₂O₂. Next, a hydrogel containing TCS/HP-β-CD inclusion (Type D) was prepared according to Example 8.

### Viscoelastic Properties

The viscoelastic properties of the hydrogels were determined using an AR-G2 rotary rheometer (TA Instruments) using the strain sweep method at a constant frequency value of about 1 Hz and a displacement between from about 10⁻³ and about 2 radians at about 25° C. In order to prevent the prepared hydrogels from slipping during the measurement, cross-hatched geometry was used. Hydrogels with a diameter of about 17.5 mm were prepared for the determination. Hydrogels were evaluated about 60 minutes after preparation, i.e. after solidification. The following table shows the viscoelastic properties of the (elastic G) hydrogels.

### Swelling

The hydrogels were immersed in saline (about 0.9% NaCl) and allowed to swell for about 24 h in an incubator at about 37° C. The hydrogels were weighed. The degree of swelling was determined on the basis of a calculation according to a formula

$$\\ Q = \frac{{m}_{s} - {m}_{o}}{{m}_{o}}$$

where Q is the swelling coefficient, mₒ is the weight of the gel after preparation and mₛ is the weight of the gel after disintegration into equilibrium.

TABLE 29
Values of elastic modulus and degree of swelling of prepared hydrogels.

| | Concentration after mixing solutions A and B | | | | | Ratio | | | |
|---|---|---|---|---|---|---|---|---|---|
| Gel | HA-TA | HRP | H₂O₂ | TCS | HP-β-CD | G′ | m₀ | mₛ | |
| type | (mg/mL) | (U/mL) | (mmol/L) | (mg/mL) | (mg/mL) | (Pa) | (mg) | (mg) | Q |
| Type A | 20 | 0.04 | 0.16 | 0 | 0 | 387 | 406 | 968 | 1.38 |
| Type B | 20 | 0.08 | 0.33 | 0 | 0 | 1156 | 427 | 590 | 0.38 |
| Type C | 20 | 0.16 | 0.65 | 0 | 0 | 3001 | 410 | 390 | −0.05 |
| Type D | 20 | 0.56 | 2.28 | 1 | 30 | 1167 | 423 | 592 | 0.4 |

### Degradation Rate

The swollen gels were transferred to other vials to which degradation medium (about 1 mL of a solution containing bovine testicular hyaluronidase with an activity of about 480 U/mL) was added. Degradation of the gels was performed at about 37° C. with stirring. During the experiment, the weight of the hydrogels was determined every 30 minutes until they were completely degraded.

### Results

Hydrogel A shows the lowest value of G′ and the highest swelling coefficient, from which it can be deduced that it also achieves the lowest degree of crosslinking. On the other hand, hydrogel C shows the highest degree of crosslinking. Although hydrogels B and D differ in the content of TCS/HP-β-CD inclusion, on the basis of comparable values of G′ and Q, it can be deduced that they also show a comparable degree of crosslinking.

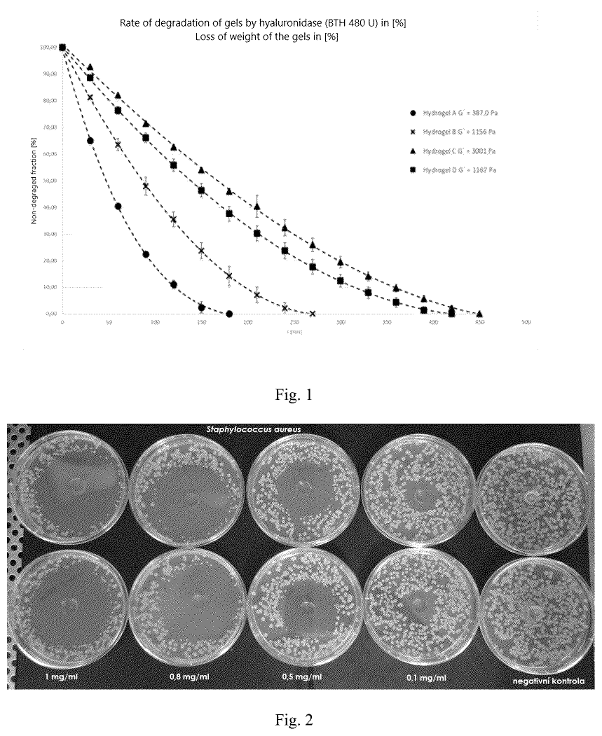

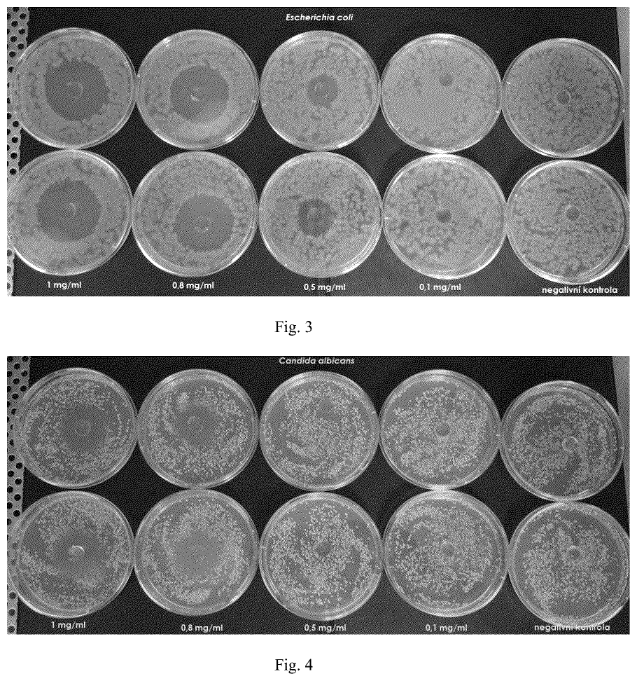

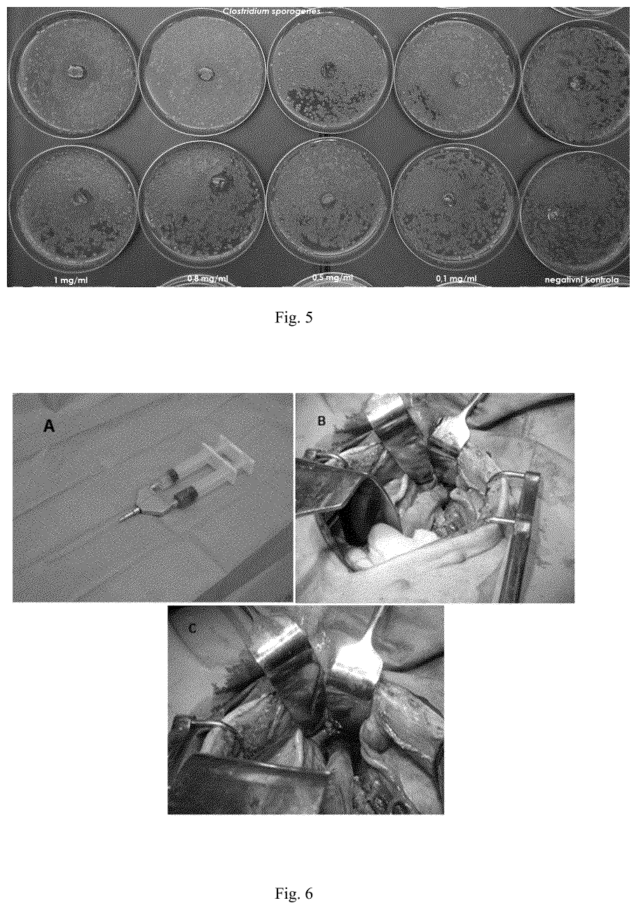

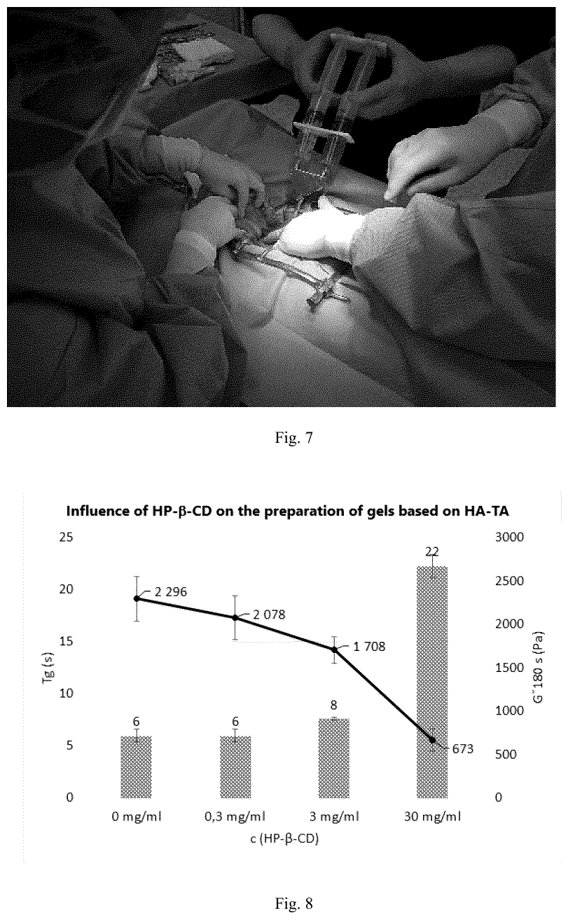

The design of the experiment allows a relative comparison of the resistance of the prepared hydrogels to the action of the hydrolytic enzyme in vitro. It is clear from the graph (see FIG. 1) that hydrogel A was the fastest to undergo enzymatic degradation, while hydrogel C was the most resistant. The results confirm that the rate of hydrogel degradation depends on the crosslink density of hydrogels, the measure thereof are viscoelastic properties of hydrogels and their degree of swelling. The rate of degradation of hydrogel D was significantly lower compared to hydrogel B, even though both types of hydrogels showed a similar degree of crosslinking. A possible cause is the presence of TCS/HP-β-CD inclusion in the structure of hydrogel D, which can slow down the enzymatic degradation of the hydrogel.

### Example 17: Effect of the Presence of HP-β-CD on the Gelation Rate and the Elastic Modulus of a Hydrogel Prepared from HA-TA

The HA-TA derivative prepared according to Example 1D was used to prepare solutions of the features for the hydrogel preparation. The concentrations of the individual components of the solution A and the solution B are given in Tables 30-33:

TABLE 30
Composition of solutions A and B for the
preparation of hydrogel I without HP-β-CD

| | |
|---|---|
| Solution A | Solution B |

| | | | | | |
|---|---|---|---|---|---|
| HRP | 0.8 | U/mL | H₂O₂ | 3.3 | mmol/L |
| HA-TA | 20 | mg/mL | HA-TA | 20 | mg/mL |

TABLE 31
Composition of solutions A and B for the preparation of
hydrogel II with a HP-β-CD content of about 0.3 mg/ml

| Solution A | | | Solution B | | |
|---|---|---|---|---|---|
| HRP | 0.8 | U/mL | H₂O₂ | 3.3 | mmol/L |
| HA-TA | 20 | mg/mL | HA-TA | 20 | mg/mL |
| Hydroxypropyl-β-cyclodextrin | 0.6 | mg/mL | | | |

TABLE 32
Composition of solutions A and B for the preparation
of hydrogel III containing HP-β-CD about 3 mg/ml

| Solution A | | | Solution B | | |
|---|---|---|---|---|---|
| HRP | 0.8 | U/mL | H₂O₂ | 3.3 | mmol/L |
| HA-TA | 20 | mg/mL | HA-TA | 20 | mg/m |
| Hydroxypropyl-β-cyklodextrin | 6 | mg/mL | | | |

TABLE 33
Composition of solutions A and B for the preparation
of hydrogel IV containing HP-β-CD about 30 mg/ml

| Solution A | | | Solution B | | |
|---|---|---|---|---|---|
| HRP | 0.8 | U/mL | H₂O₂ | 3.3 | mmol/L |
| HA-TA | 20 | mg/mL | HA-TA | 20 | mg/mL |
| Hydroxypropyl-β-cyclodextrin | 60 | mg/mL | | | |

TABLE 34
Composition and parameters of the prepared hydrogels
I to IV formed by mixing solutions A with B
according to Tables 30 to 33, as above.

| | I | II | III | VI |
|---|---|---|---|---|
| HRP (U/mL) | 0.4 | 0.4 | 0.4 | 0.4 |
| crossHA-TA (mg/mL) | 20 | 20 | 20 | 20 |
| HP-β-CD (mg/mL) | 0 | 0.3 | 3 | 30 |
| Tg (s) | 6 | 6 | 8 | 22 |
| G′_{3 min} (Pa) | 2296 | 2078 | 1708 | 673 |

### Results:

With increasing concentration of HP-β-CD in the gel-forming mixture, the rate and efficiency of the crosslinking reaction decreases, which is reflected in the prolongation of Tg and G′_{3 min}.

### Example 18: Antimicrobial Action of Triclosan-Containing Hydrogels In Vitro

### Preparation of Hydrogels for Antimicrobial Tests

The HA-TA derivative prepared according to Example 1D was used to prepare solutions of the features for the hydrogel preparation. The concentrations of the individual components of the solution A and the solution B are given in Tables 30-38:

TABLE 34
Composition of precursor solutions A and B for
the preparation of a hydrogel without TCS

| | |
|---|---|
| Solution A | Solution B |

| | | | | | |
|---|---|---|---|---|---|
| HRP | 1.13 | U/mL | H₂O₂ | 4.66 | mmol/L |
| HA-TA | 20 | mg/mL | HA-TA | 20 | mg/mL |

TABLE 35
Composition of precursor solutions A and B for the preparation
of a hydrogel with a TCS content of about 0.1 mg/mL

| Solution A | | | Solution B | | |
|---|---|---|---|---|---|
| HRP | 1.13 | U/mL | H₂O₂ | 4.66 | mmol/L |
| HA-TA | 20 | mg/mL | HA-TA | 20 | mg/mL |
| Hydroxypropyl-β-cyclodextrin | 6 | mg/mL | | | |
| Triclosan | 0.2 | mg/mL | | | |

TABLE 36
Composition of precursor solutions A and B for the preparation
of a hydrogel with a TCS content of about 0.5 mg/mL.

| Solution A | | | Solution B | | |
|---|---|---|---|---|---|
| HRP | 1.13 | U/mL | H₂O₂ | 4.66 | mmol/L |
| HA-TA | 20 | mg/mL | HA-TA | 20 | mg/mL |
| Hydroxypropyl-β-cyclodextrin | 30 | mg/mL | | | |
| Triclosan | 1 | mg/mL | | | |

TABLE 37
Composition of precursor solutions A and B for the preparation
of a hydrogel with a TCS content of about 0.8 mg/mL

| Solution A | | | Solution B | | |
|---|---|---|---|---|---|
| HRP | 1.13 | U/mL | H₂O₂ | 4.66 | mmol/L |
| HA-TA | 20 | mg/mL | HA-TA | 20 | mg/mL |
| Hydroxypropyl-β-cyclodextrin | 48 | mg/mL | | | |
| Triclosan | 1.6 | mg/mL | | | |

TABLE 38
Composition of precursor solutions A and B for the preparation
of a hydrogel with a TCS content of about 1 mg/mL

| Solution A | | | Solution B | | |
|---|---|---|---|---|---|
| HRP | 1.13 | U/mL | H₂O₂ | 4.66 | mmol/L |
| HA-TA | 20 | mg/mL | HA-TA | 20 | mg/mL |
| Hydroxypropyl-β-cyclodextrin | 60 | mg/mL | | | |
| Triclosan | 2.0 | mg/mL | | | |

Hydrogels were prepared by mixing solutions A and B in a volume ratio of about 1:1.

### Antimicrobial Activity Test

The diffusion plate method (2D arrangement) was chosen to test the effectiveness of the hydrogels. Non-selective medium—tryptone-soy agar—was used for cultivation. Blood agar was used to cultivate Clostridium sporogenes under anaerobic conditions.

Gel samples were tested in 4 microorganisms:
Staphylococcus aureus (G+cocci),
Escherichia coli (G-rods),
Clostridium sporogenes (G+anaerobic rod),
Candida albicans (yeast).

### Inoculum Preparation:

An approximately 48-hour-old culture was used to prepare the inoculum, from which a bacterial suspension of about 0.5 McFarland was prepared, corresponding to a concentration of the order of from about 10⁷-about 10⁸ CFU/mL.

In the case of yeast, a suspension of about 4.0 McFarland was prepared, which corresponds to a concentration of the order of about 10⁷ CFU/mL.

### Microbiological Analysis:

For testing itself, the suspension was further diluted to approximately 10⁴ CFU/mL and then inoculated with about 100 μL onto the surface of tryptone soy agar in Petri dishes, and the suspension was spread evenly over the surface of the entire dish with a sterile stick. The approximate number of microorganisms applied to the dish was of the order of about 10³ CFU. After soaking the suspension in the agar, the test samples were sterile transferred to its surface.

Plates with test strains and samples were stored for culture at about 37° C. for about 24 hours. Clostridium sporogenes was cultured under anaerobic conditions.

After culturing for about 24 hours, all plates inoculated with the microorganisms and test samples were evaluated. The comparison was made against a negative control, which consisted of a hydrogel without TCS/CD inclusion. A sample showing an antimicrobial effect on the test microorganism was manifested by the formation of an inhibition zone in close proximity to the test sample.

TABLE 39
Overview of the antimicrobial action of the prepared gels.

| | Tested strain | | | |
|---|---|---|---|---|
| | Staphylococcus aureus | | | |
| | Concentration TCS | | | |
| | 1 mg/mL | 0.8 mg/mL | 0.5 mg/mL | 0.1 mg/mL |
| Inhibition zone | +(58 mm) | +(55 mm) | +(40 mm) | +(20 mm) |
| | Tested strain | | | |
| | Escherichia coli | | | |
| | Concentration TCS | | | |
| | 1 mg/mL | 0.8 mg/mL | 0.5 mg/mL | 0.1 mg/mL |
| Inhibition zone | +(42 mm) | +(40 mm) | +(22 mm) | — |
| | Tested strain | | | |
| | Candida albicans | | | |
| | Concentration TCS | | | |
| | 1 mg/mL | 0.8 mg/mL | 0.5 mg/mL | 0.1 mg/mL |
| Inhibition zone | +(24 mm) | +(16 mm) | — | — |
| | Tested strain | | | |
| | Clostridium sporogenes | | | |
| | Concentration TCS | | | |
| | 1 mg/mL | 0.8 mg/mL | 0.5 mg/mL | 0.1 mg/mL |
| Inhibition zone | +(17 mm) | +(13 mm) | — | — |

*Note:
(+) The inhibition zone in close proximity to the sample/sample shows an antimicrobial effect
(−) The growth of microorganisms in the immediate vicinity of the sample does not show an antimicrobial effect

The antimicrobial effect of hydrogels on the Staphylococcus aureus strain was demonstrated for all TCS concentrations tested. The antimicrobial effect of hydrogels on Escherichia coli strain was demonstrated from a TCS concentration of about 0.5 mg/mL. The antimicrobial effect of hydrogels on the yeast Candida albicans and Clostridium sporogenes was observed only from a TCS concentration of about 0.8 mg/mL. None of the hydrogels containing TCS in the concentration range of from about 0.1 to about 1 mg/mL was colonized by bacteria, which confirms the possibility of their use as a barrier against the spread of infection.

### Example 19: Preparation of a Hydrogel by Mixing Solutions a, B and C from a Kit

The HA-TA derivative prepared according to Example 1D was used to prepare solutions of the features for the hydrogel preparation. The concentrations of the individual components of the solution A and the solution B are given in Table 40

TABLE 40
Composition of precursor solutions A, B and C for hydrogel preparation

| Solution A | | | Solution B | | Solution C | |
|---|---|---|---|---|---|---|
| HRP | 2.24 | U/mL | H₂O₂ | 9.1 mmol/L | HA-TA | 35 mg/mL |
| HA-TA | 10 | mg/mL | | | | |
| Hydroxypropyl-β-cyclodextrin | 120 | mg/mL | | | | |
| Triclosan | 4 | mg/mL | | | | |

The hydrogel was prepared by mixing solutions A, B and C in a ratio of about 1:1:2. The hydrogel thus prepared contains the enzyme horseradish peroxidase, covalently crosslinked hydroxyphenyl derivative of hyaluronan (crossHA-TA), hydroxypropyl-β-cyclodextrin and triclosan. The final composition of the hydrogel, including the values of the gelation time (Tg) and the value of the elastic module G′ after about 3 min (G′_{3 min}) and after the completion of solidification (G′ₛ) are given in Table 41.

TABLE 41
Composition and parameters of the prepared hydrogel

| | |
|---|---|
| HRP (U/mL) | 0.56 |
| crossHA-TA (mg/mL) | 20 |
| Hydroxypropyl-β-cyklodextrin (mg/mL) | 30 |
| Triclosan (mg/mL) | 1 |
| Tg (s) | 26 |
| G′_{3 min} (Pa) | 436 |
| G′ₛ (Pa) | 1040 |
| ratio TCS:HP-β-CD (mol/mol) | 1:6 |

### Example 20: Preparation of a Hydrogel by Mixing Solutions a, B and C of the Kit

The HA-TA derivative prepared according to Example 1D was used to prepare solutions of the features for the hydrogel preparation. The concentrations of the individual components of solution A and B are given in Table 42

TABLE 42
Composition of precursor solutions A, B and C for hydrogel preparation

| Solution A | | | Solution B | | Solution C | |
|---|---|---|---|---|---|---|
| HRP | 1.96 | U/mL | H₂O₂ | 9.3 mmol/L | HA-TA | 30 mg/mL |
| HA-TA | 20 | mg/mL | | | | |
| Hydroxypropyl-β-cyclodextrin | 120 | mg/mL | | | | |
| Triclosan | 4 | mg/mL | | | | |

The hydrogel was prepared by mixing solutions A, B and C in a ratio of about 1:1:2. The hydrogel thus prepared contains the enzyme horseradish peroxidase, covalently crosslinked hydroxyphenyl derivative of hyaluronan (crossHA-TA), hydroxypropyl-β-cyclodextrin and triclosan. The final composition of the hydrogel, including the values of the gelation time (Tg) and the value of the elastic module G′ after about 3 min (G′_{3 min}) and after the completion of solidification (G′ₛ) are given in Table 43.

TABLE 43
Composition and parameters of the prepared hydrogel

| | |
|---|---|
| HRP (U/mL) | 0.49 |
| crossHA-TA (mg/mL) | 20 |
| Hydroxypropyl-β-cyklodextrin (mg/mL) | 30 |
| Triclosan (mg/mL) | 1 |
| Tg (s) | 25 |
| G′_{3 min} (Pa) | 296 |
| G′ₛ (Pa) | 920 |
| ratio TCS:HP-β-CD (mol/mol) | 1:6 |

### Example 21: Preparation of a Hydrogel by Mixing Solutions a, B C of the Kit

The HA-TA derivative prepared according to Example 1D was used to prepare solutions of the features for the hydrogel preparation. The concentrations of the individual components of solution A and B are given in Table 44

TABLE 44
Composition of precursor solutions A, B and C for hydrogel preparation

| Solution A | | | Solution B | | Solution C | |
|---|---|---|---|---|---|---|
| HRP | 2.20 | U/mL | H₂O₂ | 9.4 mmol/L | HA-TA | 38 mg/mL |
| HA-TA | 5 | mg/mL | | | | |
| Hydroxypropyl-β-cyclodextrin | 120 | mg/mL | | | | |
| Triclosan | 4 | mg/mL | | | | |

The hydrogel was prepared by mixing solutions A, B and C in a ratio of about 1:1:2. The hydrogel thus prepared contains the enzyme horseradish peroxidase, covalently crosslinked hydroxyphenyl derivative of hyaluronan (crossHA-TA), hydroxypropyl-β-cyclodextrin and triclosan. The final composition of the hydrogel, including the values of the gelation time (Tg) and the value of the elastic module G′ after about 3 min (G′_{3 min}) and after the completion of solidification (G′ₛ) are given in Table 45.

TABLE 45
Composition and parameters of the prepared hydrogel

| | |
|---|---|
| HRP (U/mL) | 0.55 |
| crossHA-TA (mg/mL) | 20 |
| Hydroxypropyl-β-cyclodextrin (mg/mL) | 30 |
| Triclosan (mg/mL) | 1 |
| Tg (s) | 28 |
| G′_{3 min} (Pa) | 310 |
| G′ₛ (Pa) | 1058 |
| ratio TCS:HP-β-CD (mol/mol) | 1:6 |

### Example 22: Preclinical Testing on a Porcine Colorectal Anastomosis Dehiscence Model

The in vivo study was divided into two phases. In the first phase of in vivo tests, a hydrogel of a defined composition was implanted in the small pelvic area. The aim was to determine the relationship between the gelation time (Tg) and determined by rheological measurement and the time required to solidify (T_{solid}) the appropriate amount (V_{gel}) of material in vivo (see Table 46). T_{solid} does not agree with the in vitro determined Tg, because it does not describe the moment of hydrogel formation, but the moment when a macroscopically homogeneous hydrogel filling the small pelvic region is obtained in vivo. The hydrogel prepared according to Example 2 was used for this experiment.

TABLE 46
Parameters of the hydrogel implanted in the small pelvic area

| | Solution pA | Solution B | | | | |
|---|---|---|---|---|---|---|
| c HA-TA | HRP | H₂O₂ | TCS | T_{g} | V_{gel} | T_{solid}. |
| (mg/mL) | (U/mL) | (mM) | (mg/mL) | (s) | (mL) | (min) |
| 20 | 0.4 | 1.66 | 1 | 65 | 40 | ≈5 |

The second phase examined the effect of the presence of the hydrogel on the healing process of the colorectal anastomosis dehiscence model, which was created by perforation of the intestinal wall near the anastomosis. The severity of the condition was simulated by the size of the perforation in the range of from about 5-about 15 mm. The hydrogels of Examples 3 to 8, the properties of which are summarized in Table 47, were used to fill the small pelvic area.

TABLE 47
Parameters of hydrogels implanted in the small pelvic region
after creating a model of colorectal anastomosis dehiscence.

| | | | | | | |
|---|---|---|---|---|---|---|
| Example | 3 | 4 | 5 | 6 | 7 | 8 |

Solution A

| | | | | | | |
|---|---|---|---|---|---|---|
| HA-TA (DS %/10³ g/mol) | 2.1/294 | 2.1/294 | 2.1/294 | 2.1/294 | 2.1/294 | 2.1/294 |
| HA-TA (mg/mL) | 20 | 20 | 20 | 20 | 20 | 20 |
| Horseradish peroxidase (U/mL) | 1.2 | 0.8351 | 1.08 | 1.171 | 1.311 | 1.13 |
| HP-β-CD (mg/mL) | 60 | 60 | 60 | 60 | 60 | 60 |
| Triclosan (mg/mL) | 2.04 | 2 | 2.02 | 1.88 | 1.87 | 1.86 |

Solution B

| | | | | | | |
|---|---|---|---|---|---|---|
| HA-TA (DS %/10³ g/mol) | 2.1/294 | 2.1/294 | 2.1/294 | 2.1/294 | 2.1/294 | 2.1/294 |
| HA-TA (mg/mL) | 20 | 20 | 20 | 20 | 20 | 20 |
| Hydrogen Peroxide (mmol/L) | 4.66 | 4.66 | 4.66 | 4.66 | 4.66 | 4.66 |

Hydrogel

| | | | | | | |
|---|---|---|---|---|---|---|
| HRP (U/mL) | 0.6 | 0.418 | 0.54 | 0.59 | 0.66 | 0.56 |
| TCS (mg/mL) | 1.02 | 1 | 1.01 | 0.94 | 0.94 | 0.93 |
| G′_{3 min} (Pa) | 440 | 290 | 430 | 394 | 401 | 293 |
| Tg (s) | 28 | 38 | 28 | 31 | 30 | 36 |
| G′ₛ (Pa) | 1118 | 1051 | 1122 | 1126 | 1127 | 1055 |
| T_{solid} (min) | <5 | <5 | <5 | <5 | <5 | <5 |

At 14 days postoperatively, the animals were sacrificed and the healing status of the anastomosis was assessed. The results were evaluated by macroscopic and histological examination. An analogy with the classification of manifestations of complications associated with the healing of colorectal anastomosis in human medicine was used to evaluate the clinical condition:

TABLE 48
Definition of the severity degrees of clinical
manifestations of postoperative complications
associated with colorectal anastomosis healing.

| | Degree A | Degree B | Degree C |
|---|---|---|---|
| Clinical | Good | Minor discomfort | Serious state |
| condition | | | |
| Symptoms | no | Abdominal pain | Peritonitis |
| | | Elevated temperature | Sepsis |
| | | Purulent stools or | |
| | | rectal or vaginal | |
| | | discharge (in case of | |
| | | rectovaginal fistula) | |
| Wound | Serous fluid | Turbid fluid, | Exudate containing |
| production | maximally | possibly containing | pus |
| | slightly | pus | |
| | turbid | | |
| Laboratory | Normal | Leukocytosis, | Leukocytosis, |
| examination | | elevated level of C- | elevated level of C- |
| | | reactive protein | reactive protein |
| | | | In the case of sepsis |
| | | | development of |
| | | | leukopenia |
| Radiological | Minimal | Leakage of | Leakage of |
| examination | leakage of | anastomosis with | anastomosis and |
| | anastomosis | local complications | generalized |
| | | (absec in the pelvic | complications |
| | | region) | (peritonitis) |
| Specific | No | Treatment with | Re-operation with |
| treatment | intervention | antibiotics. | the aim to get the |
| | | Necessary to provide | infection site under |
| | | drainage | control |
| | | Transanal lavage | |

The preclinical study performed included a total of about 21 pigs with a model of colorectal anastomosis with varying degrees of damage. The onset of dehiscence was simulated by perforation of the colon near the anastomosis. The hydrogel (composition according to Table 41; from about 20 to about 40 mL/animal) was applied to about 18 animals at the end of the procedure. The condition of the animals was evaluated for about 14 days, after which they were sacrificed. In some cases, it was possible to identify gel residues at the application site even after two weeks. It is completely absorbed in less than about 30 days.

In none of the 18 cases when the hydrogel was applied, there were clinical signs of the development of sepsis, or signs of intestinal obstruction, or other side effects of the use of the developed hydrogel. The clinical condition of the animals was assessed by classification A. In contrast, in two of the three animals in the control group, in which the gel was not used during the operation, there were complications when it was necessary to use additional antibiotic treatment. The condition of these animals was classified as category B.

TABLE 49
Phase 2 evaluation of a preclinical study
Tested group

| | | | Histology | | | | |
|---|---|---|---|---|---|---|---|
| | | Perfor. | Mucosa | | serosis | | Clinical |
| Hydrogel | Anast. | (mm) | Macros. | Hist | Macros. | Hist | picture |
| HA-TA/TCS | yes | 5 | Y | Y | Y | Y | A |
| HA-TA/TCS | yes | 5 | N | N | N | Y | A |
| HA-TA/TCS | yes | 5 | Y | Y | Y | Y | A |
| HA-TA/TCS | yes | 5 | N | N | Y | Y | A |
| HA-TA/TCS | yes | 5 | Y | Y | Y | Y | A |
| HA-TA/TCS | yes | 5 | N | N | N | N | A |
| HA-TA/TCS | yes | 10 | N | N | N | N | A |
| HA-TA/TCS | yes | 10 | N | N | N | Y | A |
| HA-TA/TCS | yes | 10 | N | N | N | N | A |
| HA-TA/TCS | yes | 10 | N | N | N | N | A |
| HA-TA/TCS | yes | 10 | N | N | N | Y | A |
| HA-TA/TCS | yes | 10 | N | N | Y | Y | A |
| HA-TA/TCS | yes | 10 | N | N | N | Y | A |
| HA-TA/TCS | yes | 15 | N | N | N | N | A |
| HA-TA/TCS | yes | 15 | N | N | N | Y | A |
| HA-TA/TCS | yes | 15 | N | Y | N | Y | A |
| HA-TA/TCS | yes | 15 | N | N | N | Y | A |
| HA-TA/TCS | yes | 15 | N | N | N | Y | A |
| HA-TA/TCS | yes | 15 | Y | Y | Y | Y | A |
| HA-TA/TCS | yes | 15 | N | N | Y | Y | A |
| | | No finding | 17 | 16 | 14 | 6 | 21 |
| | | Finding | 4 | 5 | 7 | 15 | 0 |

CONTROL

| | | | Histology | | | | |
|---|---|---|---|---|---|---|---|
| | | | Mucosa | | Serosis | | Clinical |
| Hydrogel | Anast. | Perfor. | Macros. | Hist | Macros. | Hist | picture |
| No | yes | 10 | N | Y | N | Y | A |
| No | yes | 10 | N | N | N | N | B |
| No | yes | 10 | N | N | N | Y | B |
| | | No | 3 | 2 | 3 | 1 | 1 |
| | | finding | | | | | |
| | | Finding | 0 | 1 | 0 | 2 | 2 |

N—no finding
Y—macroscopic or histological finding

1. A kit of gel-forming solutions for a preparation of a biodegradable hydrogel based on a covalently crosslinked hydroxyphenyl derivative of hyaluronan, the kit comprising at least two aqueous solutions A and B, wherein the solution A comprises horseradish peroxidase and the solution B comprises hydrogen peroxide; wherein at least one of the solution A and/or the solution B comprises a hydroxyphenyl derivative of a hyaluronan having the general formula I: 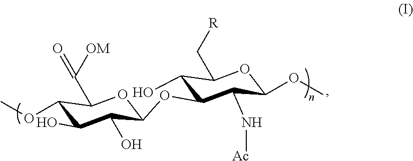
wherein n is from 2 to 5000, each M is H⁺ or a cation of a pharmaceutically acceptable salt selected from the group of alkali metal cations and alkaline earth metal cations, and each R is independently —OH or a substituent of having the general formula II: 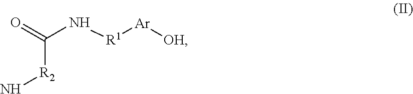
where Ar is phenylene and R₁ is ethylene, or Ar is indolydene and R₁ is ethylene, or Ar is hydroxyphenylene and R₁ is carboxyethylene, and where R₂ is an alkylene group having from 3 to 7 carbon atoms; and wherein the solution A and/or the solution B comprises triclosan and hydroxypropyl-β-cyclodextrin.

2. The kit of claim 1, wherein: (i) the horseradish peroxidase comprises an activity of from 0.5 to 1.5 U/mL: (ii) the concentration of hydrogen peroxide is from 1 to 6 mmol/L (iii) the hydroxyphenyl derivative of hyaluronan of the general formula I has a weight average molar mass of from 60,000 to 2,000,000 g/mol, a degree of substitution from 1 to 10%, and is present in a concentration of from 10 to 50 mg/mL; (iv) the concentration of triclosan is from 0.2 to 2.2 mg/mL; (v) the concentration of hydroxypropyl-β-cyclodextrin is from 4 to 100 mg/mL; and (vi) the molar ratio of triclosan to hydroxypropyl-β-cyclodextrin is from 1:4 to 1:10.

3. The kit of claim 1, wherein:
the solution A comprises:
a hydroxyphenyl derivative of hyaluronan according to the general formula I, with a weight average molar mass of from 60,000 to 2,000,000 g/mol, a polydispersity index of from 1 to 3, and a degree of substitution of from 1% to 10%, present in the solution A at a concentration of from 10 to 50 mg/mL,
a horseradish peroxidase having an activity of from 0.5 to 1.5 U/mL,
triclosan, present in the solution A at a concentration of from 0.2 to 2.2 mg/mL, and
hydroxypropyl-β-cyclodextrin, present in the solution A at a concentration of from 4 to 100 mg/mL, where the molar ratio of triclosan to hydroxypropyl-β-cyclodextrin is from 1:4 to 1:10; and
the solution B comprises:
a hydroxyphenyl derivative of hyaluronan according to the general formula I, with a weight average molar mass of from 60,000 to 2,000,000 g/mol, a polydispersity index of from 1 to 3, and a degree of substitution of from 1 to 10%, present in the solution B in a concentration of from 10 to 50 mg/mL, and
hydrogen peroxide, present in the solution B in a concentration of from 1 to 6 mmol/L.

4. The kit of claim 1,
wherein:
the solution A comprises:
a hydroxyphenyl derivative of hyaluronan according t the general formula I, with a weight average molar mass of from 100,000 to 1,000,000 g/mol, a polydispersity index of from 1 to 3, and a degree of substitution of from 1 to 5%, present in the solution A at a concentration of from 15 to 25 mg/mL,
a horseradish peroxidase having an activity of from 0.9 to 1.35 U/mL,
triclosan, present in the solution A at a concentration of from 1 to 2.2 mg/mL, and
hydroxypropyl-β-cyclodextrin, present in the solution A at a concentration of from 25 to 80 mg/mL, where the molar ratio of triclosan to hydroxypropyl-β-cyclodextrin is from 1:5 to 1:8; and
the solution B comprises:
a hydroxyphenyl derivative of hyaluronan according the general formula I, with a weight average molar mass of from 100,000 to 1,000,000 g/mol, a polydispersity index of from 1 to 3, and a degree of substitution of from 1 to 5%, present in the solution B in a concentration of from 15 to 25 mg/mL, and
hydrogen peroxide, present in the solution B in a concentration of from 3 to 5 mmol/L.

5. (canceled)

6. The kit of claim 1, further comprising, at least one solution C, wherein the solution A comprises a horseradish peroxidase, a hydroxyphenyl derivative of hyaluronan of the general formula I, triclosan, and hydroxypropyl-β-cyclodextrin; wherein the solution B comprises hydrogen peroxide; and wherein the solution C comprises a hydroxyphenyl derivative of a hyaluronan of the general formula I.

7. The kit of claim 6, wherein: (i) the horseradish peroxidase activity in the solution A is from 1 to 3 U/mL; (ii) the concentration of hydrogen peroxide in the solution B is from 2 to 12 mmol/L; (ii) in the solution A, the hydroxyphenyl derivative of hyaluronan according to the general formula I has a weight average molar mass of from 60,000 to 2,000,000 g/mol, a degree of substitution of from 1 to 10%, and is present in the solution A at a concentration of from 1 to 20 mg/mL; (iv) in the solution C, the hydroxyphenyl derivative of hyaluronan according to the general formula I has a weight average molar mass of from 60,000 to 2,000,000 g/mol, a degree of substitution of from 1 to 10%, and is present in the solution C at a concentration of from 10 to 50 mg/mL; (v) the concentration of triclosan in the solution A is from 0.2 to 4.4 mg/mL, (vi) the concentration of hydroxypropyl-β-cyclodextrin in the solution A is from 8 to 200 mg/mL; and (vii) the molar ratio of triclosan to hydroxypropyl-β-cyclodextrin in the solution A is from 1:4 to 1:10.

8. The kit
of claim 6, wherein:
the solution A comprises:
a hydroxyphenyl derivative of hyaluronan according the general formula I, with a weight average molar mass of from 60,000 to 2,000,000 g/mol, a polydispersity index of from 1 to 3, and a degree of substitution of from 1 to 10%, present in the solution A at a concentration of from 1 to 20 mg/mL,
a horseradish peroxidase having an activity of from 1 to 3 U/mL,
triclosan, present in the solution A at a concentration of from 0.4 to 4.4 mg/mL, and
hydroxypropyl-β-cyclodextrin, present in the solution A at a concentration of from 8 to 200 mg/mL, where the molar ratio of triclosan to hydroxypropyl-j-cyclodextrin is from 1:4 to 1:10;
the solution B comprises:
hydrogen peroxide, present in the solution B in a concentration of from 2 to 12 mmol/L;
and
the solution C comprises:
a hydroxyphenyl derivative of hyaluronan according the general formula I, with a weight average molar mass of from 60,000 to 2,000,000 g/mol, a polydispersity index of from 1 to 3, and a degree of substitution of from 1 to 10%, present in the solution C in a concentration of from 10 to 50 mg/mL.

9. The kit
of claim 6, wherein:
the solution A comprises:
a hydroxyphenyl derivative of hyaluronan according to the general formula I, with a weight average molar mass of from 200,000 to 400,000 g/mol, a polydispersity index of from 1 to 3, and a degree of substitution of from 2 to 4%, present in the solution A at a concentration of from 5 to 15 mg/mL,
a horseradish peroxidase having an activity of from 1.6 to 2.7 U/mL,
triclosan, present in the solution A at a concentration of from 3.6 to 4.4 mg/mL, and
hydroxypropyl-β-cyclodextrin, present in the solution A at a concentration of from 108 to 132 mg/mL, where the molar ratio of triclosan to hydroxypropyl-β-cyclodextrin is from 1:4 to 1:10;
the solution B comprises:
hydrogen peroxide, present in the solution B in a concentration of from 7 to 12 mmol/L; and
the solution C comprises:
a hydroxyphenyl derivative of hyaluronan according the general formula I, with a weight average molar mass of from 60,000 to 2,000,000 g/mol, a polydispersity index of from 1 to 3, and a degree of substitution of from 1 to 10%, present in the solution C in a concentration of from 30 to 40 mg/mL.

10. (canceled)

11. (canceled)

12. A method of preparing a hydrogel containing a covalently crosslinked hydroxyphenyl derivative of hyaluronan intended for prevention of postoperative complications associated with colorectal anastomosis from the kit of claim 1, comprising:
preparing separately the at least two solutions A and B; and
mixing together the solution A with the solution B to form a hydrogel containing a covalently crosslinked hydroxyphenyl derivative of hyaluronan.

13. The method of claim 12, wherein the solution A contains horseradish peroxidase with an activity of from 0.5 to 1.5 U/mL and the solution B contains hydrogen peroxide in a concentration of from 1 to 6 mmol/L; and wherein the solution A and/or the solution B contains a hydroxyphenyl derivative of hyaluronan of the general formula I having a weight average molar weight of from 60,000 to 2,000,000 g/mol, a degree of substitution of from 1 to 10%, at a concentration of from 10 to 50 mg/mL.

14. The method of claim 13, wherein the solution A and/or the solution B comprises a concentration of triclosan of from 0.2 to 2.2, a concentration of hydroxypropyl-β-cyclodextrin of from 4 to 100 mg/mL, and a molar ratio of triclosan to hydroxypropyl-β-cyclodextrin of from 1:4 to 1:10.

15. A method of preparing a hydrogel containing a covalently crosslinked hydroxyphenyl derivative of hyaluronan intended for prevention of postoperative complications associated with colorectal anastomosis from the kit of claim 3, comprising:
preparing separately the at least two solutions A and B; and
mixing together the solution A with the solution B to form a hydrogel Containing a covalently crosslinked hydroxyphenyl derivative of hyaluronan.

16. A method of preparing a hydrogel containing a covalently crosslinked hydroxyphenyl derivative of hyaluronan intended for prevention of postoperative complications associated with colorectal anastomosis from the kit of claim 4, comprising:
preparing separately the at least two solutions A and B; and
mixing together the solution A with the solution B to form a hydrogel containing a covalently crosslinked hydroxyphenyl derivative of hyaluronan.

17. (canceled)

18. (canceled)

19. A method of preparing hydrogel containing a covalently crosslinked hydroxyphenyl derivative of hyaluronan intended for prevention of postoperative complications associated with colorectal anastomosis from the kit of claim 6, comprising:
preparing separately the at least two solutions A and B and the solution C; and
mixing together the solution A with the solution B and the solution C to form a hydrogel containing a covalently crosslinked hydroxyphenyl derivative of hyaluronan.

20. A method of preparing a hydrogel containing a covalently crosslinked hydroxyphenyl derivative of hyaluronan intended for prevention of postoperative complications associated with colorectal anastomosis from the kit of claim 7, comprising:
preparing separately the at least two solutions A and B and the solution C; and
mixing together the solution A with the solution B and the solution C to form a hydrogel containing a covalently crosslinked hydroxyphenyl derivative of hyaluronan.

21. The method of claim 19, wherein the solutions A, B and C are mixed together in a volume ratio of about 1:1:2.

22. The method of claim 12, wherein after mixing at least two solutions A and B, the hydrogel containing a covalently crosslinked hydroxyphenyl derivative of hyaluronan 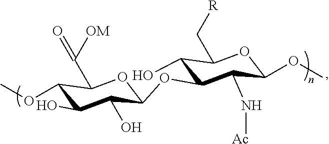 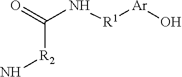
reaches a gelation point within 5 to 70 s, with a value of elastic module of from 100 to 1000 Pa, and no later than 3 min after mixing the solutions an elastic module of from 100 to 600 Pa, and upon completion of the solidification process an elastic module of from 500 to 2000 Pa.

23. A hydrogel prepared according to the method of claim 12.

24. A hydrogel prepared according to the method of claim 19.

25. A hydrogel prepared according to the method of claim 22.

26. (canceled)
